SEQUENCE LISTING (46 sequences)

SEQ ID NO: 1 - protein, 14 aa, Homo sapiens
GILEDMPVDPDNEA

SEQ ID NO: 2 - protein, 14 aa, synthetic construct
GGKESMPVDPDNEA

SEQ ID NO: 3 - protein, 14 aa, synthetic construct
GGKEDMPVDPDNEA

SEQ ID NO: 4 - protein, 12 aa, synthetic construct
LESMPVDPDNES

SEQ ID NO: 5 - protein, 12 aa, synthetic construct
LEKMPVDPDNEA

SEQ ID NO: 6 - protein, 14 aa, synthetic construct
GGSESMPVDPDNEA

SEQ ID NO: 7 - protein, 14 aa, synthetic construct
GGSESMPVDPDNES

SEQ ID NO: 8 - protein, 12 aa, synthetic construct
KEDMPVDPDNEA

SEQ ID NO: 9 - protein, 12 aa, synthetic construct
SESMPVDPDNEA

SEQ ID NO: 10 - protein, 12 aa, synthetic construct
LESMPVDPDNEA

SEQ ID NO: 11 - protein, 14 aa, synthetic construct
GGKESMPVDPDNES

SEQ ID NO: 12 - protein, 12 aa, synthetic construct
KESMPVDPDNEA

SEQ ID NO: 13 - protein, 12 aa, synthetic construct
KESMPVDPDNES

SEQ ID NO: 14 - protein, 12 aa, synthetic construct
SEKMPVDPDNEA

SEQ ID NO: 15 - protein, 12 aa, synthetic construct
LEKMPVDPDNES

SEQ ID NO: 16 - protein, 14 aa, synthetic construct
GGSESMPVDPDNEK

SEQ ID NO: 17 - protein, 12 aa, synthetic construct
LEKMPVDPDNEK

SEQ ID NO: 18 - protein, 14 aa, synthetic construct
GGKEKMPVDPDNEA

SEQ ID NO: 19 - protein, 14 aa, synthetic construct
GGKESMPVDPDNEK

SEQ ID NO: 20 - protein, 14 aa, synthetic construct
GGKEKMPVDPDNEK

SEQ ID NO: 21 - protein, 20 aa, Corynebacterium diphtheriae
VHHNTEEIVAQSIALSSLMV

SEQ ID NO: 22 - protein, 25 aa, Influenza A virus
IDGVKLESMGVYQILAIYSTVASSL

SEQ ID NO: 23 - protein, 20 aa, Human gammaherpesvirus 4
VYGGSKTSLYNLRRGTALAI

SEQ ID NO: 24 - protein, 13 aa, Influenza A virus
SMGVYQILAIYST

SEQ ID NO: 25 - protein, 10 aa, Corynebacterium diphtheriae
IVAQSIALSS

SEQ ID NO: 26 - protein, 10 aa, Clostridium tetani
YIKANSKFIG

SEQ ID NO: 27 - protein, 10 aa, Human gammaherpesvirus 4
LYNLRRGTAL

SEQ ID NO: 28 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 29 - protein, 40 aa, synthetic construct
VHHNTEEIVAQSIALSSLMVPMGAPQYIKANSKFIGITEL

SEQ ID NO: 30 - protein, 57 aa, synthetic construct
VYGGSKTSLYNLRRGTALAIVVRQYIKANSKFIGITELVVRPIFFLHHSNTDRLWAI

SEQ ID NO: 31 - protein, 38 aa, synthetic construct
VYGGSKTSLYNLRRGTALAIVVRQYIKANSKFIGITEL

SEQ ID NO: 32 - protein, 52 aa, synthetic construct
SMGVYQILAIYSTVVRIVAQSIALSSVVRYIKANSKFIGVVRLYNLRRGTAL

SEQ ID NO: 33 - protein, 52 aa, synthetic construct
SAGVYQILAIYSTVVRIVAQSIALSSVVRYIKANSKFIGVVRLYNLRRGTAL

SEQ ID NO: 34 - DNA, 24 bp, synthetic construct
  source 1..24: mol_type = other DNA
tcgtcgttttgtcgttttgtcgtt

SEQ ID NO: 35 - protein, 13 aa, synthetic construct
AKFVAAWTLKAAA

SEQ ID NO: 36 - protein, 15 aa, synthetic construct
QYIKANSKFIGITEL

SEQ ID NO: 37 - protein, 21 aa, synthetic construct
FNNFTVSFWLRVPKVSASHLE

SEQ ID NO: 38 - protein, 12 aa, Homo sapiens
LEDMPVDPDNEA

SEQ ID NO: 39 - protein, 12 aa, synthetic construct
LESSPVDPDNEA

SEQ ID NO: 40 - protein, 12 aa, synthetic construct
SEDMPVDPDNES

SEQ ID NO: 41 - protein, 12 aa, synthetic construct
LEDSPVDPDNES

SEQ ID NO: 42 - protein, 12 aa, synthetic construct
SEDSPVDPDNEA

SEQ ID NO: 43 - protein, 7 aa, synthetic construct
DQPVLPD

SEQ ID NO: 44 - skipped

SEQ ID NO: 45 - protein, 14 aa, synthetic construct
GGLEDMPVDPDNEA

SEQ ID NO: 46 - protein, 13 aa, synthetic construct
SAGVYQILAIYST

(19) United States
(12) Patent Application Publication
(10) Pub. No.: US 2025/0152684 A1
(43) Pub. Date: May 15, 2025

(54) Anti-Alpha-Synuclein Therapeutic Vaccines

(71) Applicant: AC Immune SA, Lausanne (CH)

(72) Inventors: Andrea Pfeifer, Lausanne (CH); Maxime Ayer, Lausanne (CH)

(21) Appl. No.: 18/836,298

(22) PCT Filed: Feb. 9, 2023
(86) PCT No.: PCT/EP2023/053259

§ 371 Date: Aug. 6, 2024

(30) Foreign Application Priority Data
Feb. 9, 2022 (EP) 22156006.3
Feb. 9, 2022 (EP) 22156007.1

(51) Int. Cl. A61K 39/00; A61K 9/127; A61K 39/385; A61P 25/28

(52) U.S. Cl. CPC A61K 39/0007; A61K 9/127; A61K 39/385; A61P 25/28; A61K 2039/55555; A61K 2039/55561; A61K 2039/55572; A61K 2039/6037

(57) ABSTRACT

The present invention relates to a liposomal vaccine composition comprising: a peptide antigen displayed on the surface of the liposome; a peptide comprising a T-cell epitope; and an adjuvant; wherein the peptide antigen comprises, consists essentially of or consists of the structure: X₁-X₂-X₃-E-X₄-X₅-P-V-D-P-D-N-E-X₆, wherein: E is glutamic acid, P is proline; V is Valine, D is aspartic acid, N is asparagine; X₁ is present or not and, if present, is G, wherein G is glycine; X₂, is present or not and, if present, is G, wherein G is defined as above; X₃ is L, K, or S, wherein L is leucine, K is lysine, and S is serine; X₄ is D, K or S, wherein D, K and S are as defined above; X₅ is M, wherein M is methionine or methionine sulfoxide; X₆ is A, K or S, wherein A is alanine and K, and S are as defined above; with the proviso that X₃-E-X₄-X₅-P-V-D-P-D-N-E-X₆ is not L-E-D-M-P-V-D-P-D-N-E-A, and which comprises between 1 and 5 amino acid differences compared with the amino acid sequence G-I-L-E-D-M-P-V-D-P-D-N-E-A, and wherein the peptide antigen does not comprise the dipeptide Y-E immediately following X₆, wherein Y is tyrosine and E is as defined above.

### FIELD OF THE INVENTION

The present invention relates to anti-alpha-synuclein therapeutic vaccines that can be employed for the prevention, alleviation and/or treatment of diseases, disorders and abnormalities associated with alpha-synuclein (α-synuclein, A-synuclein, aSynuclein, A-syn, α-syn, aSyn, a-syn) aggregates including, but not limited to, Lewy bodies and/or Lewy neurites and/or glial cytoplasmic inclusions, such as Parkinson's disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease.

### BACKGROUND OF THE INVENTION

Many degenerative diseases are associated with extracellular or intracellular deposits of amyloid or amyloid-like proteins that contribute to the pathogenesis as well as to the progression of the disease. The best characterized amyloid protein that forms extracellular aggregates is amyloid beta (A3). Amyloid-like proteins that form mainly intracellular aggregates, include, but are not limited to alpha-synuclein, tau, and huntingtin (htt).

aSyn is a 14 kD naturally monomeric protein that is normally located to presynaptic terminals either bound to membranes of the synaptic vesicles or in the cytosol. Its natural function remains poorly understood and is likely involved in the synaptic transmission. During pathogenesis misfolding and aggregation of aSyn occurs in the central nervous system (CNS) and the peripheral nervous system, possibly as a consequence of posttranslational modification, including among others C-terminal protease cleavage (Dufty 2007, Bassil 2016). Aggregation leads to the generation of different aSyn species that have been associated with the pathogenesis of LB diseases, such as oligomers, protofibrils, and fibrils. The fibrillar forms of aSyn are detected mostly in LBs which are located in neuronal cell body (Kosaka et al., 1990, Dickson et al, 1989). Aggregates of aSyn can be also detected in astroglial cells (Braak 2007). Not only fibrils, but various oligomeric aSyn species were detected in diseased human brains. In contrast to fibrillar aSyn, oligomeric aggregates are most likely located in neuronal projections and presynaptic terminals where they might damage the synapses, thus oligomeric aSyn has been attributed to cellular cytotoxicity.

It has been shown that monomeric aSyn can form different types of aggregates with different appearances, conformations, cytotoxicities and chemical properties under different in vitro conditions. Depending on the conformation of the monomer and the pre-vailing permissive conditions, different types of aggregates can develop, possessing different structural characteristics. When seeded, distinct aSyn-strains impress (e.g. “fibrils” or “ribbons”) their conformation upon the receiving cell and generate aggregates of the same strain in a process termed “conformational templating”. If they are injected into rat brains, these types of aggregates show varying properties in terms of inclusion formation and generation of behavioral and neurotoxic phenotypes in vivo.

It is reasoned that different types of aSyn aggregates expose different polypeptide chains due to their distinct conformations. These differentially exposed surfaces would allow different sets of intramolecular interactions. Thus, the conformation of a given aSyn-strain dictates its properties such as their propensity for seeding or the predilection for certain cell types. Experimental data start to emerge that demonstrate the different properties of aSyn-strains extracted from PD and Multiple System Atrophy (MSA) material; Analysis of pathologic brain material from patients with PD or MSA demonstrated different properties of transmissible aSyn aggregates.

Diseases involving alpha-synuclein aggregates are generally listed as synucleinopathies (or α-synucleinopathies, or alpha-synucleinopathies) and these include, but are not limited to, Parkinson's disease (PD). Synucleinopathies include Parkinson's disease (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), diffuse Lewy body disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, and Down syndrome. Synucleinopathies with neuronal and glial aggregates of alpha-synuclein include but are not limited to multiple system atrophy (Shy-Drager syndrome, striatonigral degeneration and olivopontocerebellar atrophy). Other diseases that may have alpha-synuclein-immunoreactive lesions include traumatic brain injury, chronic traumatic encephalopathy, dementia puglistica, tauopathies (Pick's disease, frontotemporal dementia, progressive supranuclear palsy, corticobasal degeneration and Niemann-Pick type C1 disease, frontotemporal dementia with Parkinsonism linked to chromosome 17), motor neuron disease, Huntington's disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Creutzfeldt-Jakob disease, ataxia telangiectatica, Meige's syndrome, subacute sclerosing panencephalitis, Gerstmann-Straussler-Scheinker disease, inclusion-body myositis, Gaucher disease, Krabbe disease as well as other lysosomal storage disorders (including Kufor-Rakeb syndrome and Sanfilippo syndrome) and rapid eye movement (REM) sleep behavior disorder (Jellinger, Mov Disord 2003, 18 Suppl. 6, S2-12; Galvin et al., JAMA Neurology 2001, 58 (2), 186-190; Kovari et al., Acta Neuropathol. 2007, 114(3), 295-8; Saito et al., J Neuropathol Exp Neurol. 2004, 63(4), 323-328; McKee et al., Brain, 2013, 136(Pt 1), 43-64; Puschmann et al., Parkinsonism Relat Disord 2012, 18S1, S24-S27; Usenovic et al., J Neurosci. 2012, 32(12), 4240-4246; Winder-Rhodes et al., Mov Disord. 2012, 27(2), 312-315; Ferman et al., J Int Neuropsychol Soc. 2002, 8(7), 907-914; Smith et al., J Pathol. 2014; 232:509-521, Lippa et al., Ann Neurol. 1999 March; 45(3):353-7; Schmitz et al., Mol Neurobiol. 2018 Aug. 22; Charles et al., Neurosci Lett. 2000 Jul. 28; 289(1):29-32; Wilhelmsen et al., Arch Neurol. 2004 March; 61(3):398-406; Yamaguchi et al., J Neuropathol Exp Neurol. 2004, 80^{th} annual meeting, vol. 63; Askanas et al., J Neuropathol Exp Neurol. 2000 July; 59(7):592-8).

Parkinson's Disease (PD) is a synucleinopathy and the second most common neurodegenerative movement disease. PD prevalence ranges between 100 and 200/100,000 in the general population, and affects approximately 1% of the population above the age of 60 with an annual incidence of about 15/100,000. It is a chronic progressive disorder, defined by a combination of motoric syndromes (bradykinesia, rigidity, resting tremor and postural instability) and by non-motoric syndromes (a variety of autonomic dysfunctions, sensory abnormalities, and psychiatric abnormalities) that usually precede motoric syndromes. The hallmark of the disease is a profound loss of dopaminergic neurons in the substantia nigra (SN), accompanied by the accumulation of filamentous protein inclusions, termed Lewy Bodies (LB), which are predominantly composed of alpha-synuclein (aSyn). PD, DLB and other LB diseases show accumulation and redistribution of aSyn in various brains regions and cellular populations.

MSA is another very important synucleinopathy. MSA is a sporadic neurodegenerative disorder that is characterized by symptoms of L-DOPA-resistant parkinsonism, cerebellar ataxia, and dysautonomia. Patients suffer from multisystem neuronal loss affecting various brain areas including striatum, substantia nigra, cerebellum, pons, as well as the inferior olives and the spinal cord. MSA is characterized by aSyn-positive glial cytoplasmic (GCI) and rare neuronal inclusions throughout the central nervous system. These inclusions are associated with striatonigral degeneration, olivopontocerebellar atrophy, and involvement of autonomic nuclei in medulla and spinal cord. The importance of GCIs for the pathogenesis of MSA is generally acknowledged and underscored by recent analysis of transgenic mouse models analysing the effect of aSyn overexpression in oligodendroglia. In tg mice overexpressing human aSyn both GCI-like aggregates and biochemical markers of MSA were observed.

DLB is the second most common type of neurodegenerative dementias in western society after Alzheimer's disease (AD). It makes up for 4-7% of clinically diagnosed dementia, with the same number of cases predicted to escape correct clinical diagnosis. Diagnosis of DLB is challenging, as the disease represents an “in-between” of AD and PD and shows overlapping features of both entities. The four clinical consensus criteria, of which two must be present to diagnose “probable DLB” are fluctuation in cognition and attention, recurrent visual hallucinations, REM sleep behavior disorder and spontaneous parkinsonian motor signs, which occur later in the disease than the other criteria. These can be supported by a variety of additional clinical criteria that can, but need not occur, such as syncope and transient episodes of un-responsiveness, apathy, anxiety, depressions, psychotic episodes and neuroleptic sensitivity and many others. Symptoms are not uniform among patients.

DLB pathology is characterized by proteinaceous inclusions termed Lewy Bodies (LB), predominantly composed of alpha synuclein (aSyn) that has a role in the loss of function and structure of the neurons. However, in DLB, the LB are found distributed diffusely throughout the cortices, while in PD, they are found pre-dominantly in the dopaminergic neurons of the Substantia Nigra. The LB in DLB are less well demarcated, less eosinophilic and less filamentous than those of PD. In addition, amyloid plaques containing mainly carboxy-terminally elongated forms of amyloid beta (Abeta) such as Abeta1-42 can be found in the brains of DLB patients. Cortical amyloid deposition is associated with lower temporal lobe perfusion and a trend to hippocampal atrophy.

Current potential therapies that are able to modify the underlying neurodegeneration related to alpha-synuclein are still under development. Vaccination with PD01 and PD03, previously developed AFFITOPE® targeting aSyn, has proven efficacy in various animal models of aSyn aggregation disorders, reducing aSyn pathology, preservation of neuroinflammation, as well as amelioration of behavior deficits (Mandler et al. 2014; WO 2009/103105 A1, WO 2011/020133 A1, WO 2017/076873 A1). These peptides turned out to be safe and well-tolerated vaccines which are able to induce target-specific antibodies in humans. Another alpha synuclein targeting active immunotherapy, UB-312, was tested in a human alpha synuclein transgenic mouse model and has been shown to reduce the accumulation of alpha synuclein in the brain and gut. This was accompanied by an improvement in motor performance of treated animals (Nimmo J T et al., 2022 Acta Neuropathol, January; 143(1):55-73). UB-312 is currently being tested in a Phase 1/2 clinical trial. This study will determine the safety, tolerability, and immunogenicity of UB-312 in healthy participants and PD and MSA patients (Fleming S M et al., 2022, Neuropharmacology 202).

There are currently no approved anti-alpha-synuclein vaccines on the market to prevent and/or treat alpha-synuclein-related diseases. It may therefore be desirable to identify new therapeutic vaccine compositions that can prevent and/or treat these diseases.

### DESCRIPTION

The inventors have designed vaccine compositions comprising antigenic peptides, which are shown herein to be highly immunogenic and which induce high amounts of aSyn-specific antibodies in the periphery. The vaccine compositions are predicted to increase target binding of the induced antibodies due to an oligoclonal antibody response.

In one general aspect, the present invention relates to a liposomal composition comprising an antigenic peptide displayed on the surface of the liposome and an adjuvant, wherein the antigenic peptide has the structure:

X₁-X₂-X₃-E-X₄-X₅-P-V-D-P-D-N-E-X₆,
wherein:
E is glutamic acid, P is proline; V is Valine, D is aspartic acid, N is asparagine;
X₁, is present or not and, if present, is G, wherein G is glycine;
X₂, is present or not and, if present, is I or G, wherein I is isoleucine and G is defined as above;
X₃ is L, K, or S, wherein L is leucine, K is lysine, and S is serine;
X₄ is D, K or S, wherein D, K and S are as defined above;
X₅ is M, wherein M is methionine or methionine sulfoxide;
X₆ is A, K or S, wherein A is alanine and K and S are as defined above.

Single letter amino acid code, as commonly known in the art, is used herein.

In one aspect, the invention provides a liposomal vaccine composition comprising an antigenic peptide displayed on the surface of the liposome and an adjuvant, wherein the antigenic peptide has the structure:

X₁-X₂-L-E-D-M-P-V-D-P-D-N-E-A
wherein:
L is leucine, E is glutamic acid, M is methionine or methionine sulfoxide, P is proline; V is valine, D is aspartic acid, N is asparagine, A is alanine;
X₁, is present or not and, if present, is G, wherein G is glycine;
X₂, is present or not and, if present, is I or G, wherein I is isoleucine and G is defined as above.

In one embodiment, the invention relates to a liposomal vaccine composition comprising an antigenic peptide displayed on the surface of the liposome and an adjuvant, wherein the antigenic peptide comprises, consists essentially of, or consists of the amino acid sequence G-I-L-E-D-M-P-V-D-P-D-N-E-A (SEQ ID NO: 1). Thus, the invention in one aspect encompasses the use of a native aSyn-peptide (amino acid position 111-124, with reference to SEQ ID NO: 28) in the liposomal vaccine composition.

In one embodiment, the invention relates to a liposomal vaccine composition comprising an antigenic peptide displayed on the surface of the liposome and an adjuvant, wherein the antigenic peptide comprises, consists essentially of or consists of the amino acid sequence L-E-D-M-P-V-D-P-D-N-E-A (SEQ ID NO: 38). Thus, the invention in another aspect encompasses the use of a native aSyn-peptide (amino acid position 113-124, with reference to SEQ ID NO: 28) in the liposomal vaccine composition.

The antigenic peptide of the liposomal vaccine composition may comprise, consist essentially of, or consist of the amino acid sequence G-G-L-E-D-M-P-V-D-P-D-N-E-A (SEQ ID NO: 45). Therefore, the invention may encompass the use of aSyn-peptide (amino acid position 111-124) with one I to G substitution at amino acid position 112 where X₂ is G and therefore the aSyn-peptide of the liposomal vaccine composition has the amino acid sequence: G-G-L-E-D-M-P-V-D-P-D-N-E-A (SEQ ID NO: 45).

The antigenic peptide may optionally comprise between 1 and 2 amino acid differences (i.e. 1, 2 differences) compared with the amino acid sequence G-I-L-E-D-M-P-V-D-P-D-N-E-A. The differences are generally amino acid substitutions according to the options set out for each position. These differences can be selected from any of amino acids X₁-X₂. Absence of X₁ and/or X₂ is considered a difference. For example, SEQ ID NO: 38 is lacking amino acids in positions X₁ and X₂ and thus presents two differences compared to SEQ ID NO: 1 and SEQ ID NO: 45 presents an amino acid substitution in position X₂ (wherein I is substituted for G) and therefore presents one difference as compared to the amino acid sequence G-I-L-E-D-M-P-V-D-P-D-N-E-A.

In some embodiments the antigenic peptide of the liposomal vaccine composition comprising the amino acid sequences: SEQ ID NO: 1, SEQ ID NO: 38 or SEQ ID NO: 45, does not comprise the dipeptide Y-E immediately following the alanine residue (i.e. in position X₆), wherein Y is tyrosine and E is glutamic acid.

Typically, the antigenic peptides comprised in the liposomal vaccine composition of the invention do not comprise further alpha synuclein amino acid residues after the alanine of SEQ ID NO: 1 or SEQ ID NO: 38. In particular, they do not comprise the dipeptide Y-E immediately following the alanine.

The antigenic peptides of the liposomal vaccine compositions may comprise, consist of, or consist essentially of the amino acid sequences selected from SEQ ID NO: 1, SEQ ID NO: 38 or SEQ ID NO: 45. The antigenic peptides may, however, comprise a limited number of further N and/or C terminal amino acid residues in order to allow the insertion of the antigenic peptide into the liposome (in a manner that permits the peptide antigen to be displayed on the surface of the liposome). For example, the antigenic peptide can include additional residues, such as lysine residues to facilitate palmitoylation. Those residues are typically found at the N and/or C terminus of the antigenic peptide sequence. In some embodiments, there may be 1-4 lysine residues added to the N and/or C terminus, preferably 2 lysine residues added at the N and C terminus.

In this context, the term “consists essentially of” means that the alpha-synuclein-derived peptide antigen includes the 10 to 14 contiguous amino acids starting on position 111 or 113 of the alpha-synuclein (SEQ ID NO: 28) but can include a limited number of additional residues, such as two to four lysine residues to facilitate presentation on the surface of the liposome.

The peptide antigen comprising, consisting of, or consisting essentially of the amino acid sequences SEQ ID NO: 1, SEQ ID NO: 38 or SEQ ID NO: 45 included in the liposomal compositions of the invention may further comprise at least one chemical modification. Modifications, such as amidation, esterification, palmitoylation, formylation, acetylation, other chemical substitution etc. may be made to a free C-terminal (or N-terminal) end of a peptide or its side chains. Such modifications are within the scope of the term “consisting essentially of” as used herein even if not separately specified. Palmitoylation is a preferred modification to facilitate peptide antigen display on the surface of the liposome, which may be in a structural arrangement conducive to production of neutralizing antibodies.

Embodiments of the invention will now be defined with reference to numbered clauses.
1. A liposomal vaccine composition comprising:
A peptide antigen displayed on the surface of the liposome;
A peptide comprising a T-cell epitope; and
An adjuvant;
wherein the peptide antigen comprises, consists essentially of or consists of the structure:

X₁-X₂-L-E-D-M-P-V-D-P-D-N-E-A,
wherein:
L is leucine, E is glutamic acid, M is methionine or methionine sulfoxide, P is proline; V is valine, D is aspartic acid, N is asparagine, A is alanine;
X₁ is present or not and, if present, is G, wherein G is glycine;
X₂, is present or not and, if present, is I or G, wherein I is isoleucine and G is defined as above;
wherein the peptide antigen does not comprise the dipeptide Y-E immediately following A,
wherein Y is tyrosine and E is as defined above.
2. The liposomal composition of clause 1 wherein X₁ and X₂ are present.
3. The liposomal composition of clause 1 wherein X₁ and X₂ are absent.
4. The liposomal composition of clause 1 or 2, wherein X₂, is I.
5. The liposomal composition of clause 1 wherein the peptide antigen is selected from the group consisting of G-I-L-E-D-M-P-V-D-P-D-N-E-A (SEQ ID NO: 1), L-E-D-M-P-V-D-P-D-N-E-A (SEQ ID NO: 38) and G-G-L-E-D-M-P-V-D-P-D-N-E-A (SEQ ID NO: 45).
6. The liposomal composition of any one of the preceding clauses, wherein M is methionine sulfoxide.
7. The liposomal vaccine composition of any one of the preceding clauses, wherein the peptide antigen further comprises at least one chemical modification.
8. The liposomal vaccine composition of any one of the preceding clauses, wherein one or both ends of the peptide antigen further comprise at least one arginine I or glutamic acid I residue.
9. The liposomal vaccine composition of any one of the preceding clauses, wherein one or both ends the peptide antigen further comprise at least 3 arginine I or glutamic acid I residues.
10. The liposomal vaccine composition of any one of the preceding clauses, wherein the peptide comprising a T-cell epitope comprises at least one amino acid sequence selected from the group consisting of SEQ ID NO: 35 (PaDre), SEQ ID NO: 36 (P2), SEQ ID NO: 37 (P30), SEQ ID NO: 21 (SAT13), SEQ ID NO: 22 (SAT15), SEQ ID NO: 23 (SAT17), SEQ ID NO: 24, SEQ ID NO: 25, SEQ ID NO: 26, SEQ ID NO: 27 and any combination thereof.
11. The liposomal vaccine composition of any one of the preceding clauses, wherein the peptide comprising a T-cell epitope comprises, consists essentially of or consists of an amino acid sequence selected from the group consisting of SEQ ID NO: 29 (SAT42), SEQ ID NO: 30 (SAT43), SEQ ID NO: 31 (SAT44), SEQ ID NO: 32 (SAT47), SEQ ID NO: 35 (PaDre), SEQ ID NO: 36 (P2) and SEQ ID NO: 37 (P30), or a close sequence analogue thereof.
12. The liposomal vaccine composition of any one of the preceding clauses, wherein the peptide comprising a T-cell epitope comprises, consists essentially of or consists of the amino acid sequence of SEQ ID NO: 32 (SAT47) or a close sequence analogue thereof.
13. The liposomal vaccine composition of any one of the preceding clauses, wherein the adjuvant comprises a toll-like receptor 4 ligand and/or a toll-like receptor 9 ligand.
14. The liposomal vaccine composition of clause 13, wherein the toll-like receptor 4 ligand comprises monophosphoryl lipid A (MPLA) and/or wherein the toll-like receptor 9 ligand comprises CpG.
15. The liposomal vaccine composition of any one of the preceding clauses, wherein the adjuvant comprises monophosphoryl lipid A (MPLA).
16. The liposomal vaccine composition of any one of the preceding clauses, wherein the adjuvant further comprises CpG.
17. The liposomal vaccine composition of any one of the preceding clauses, wherein the peptide comprising a T-cell epitope is encapsulated in the liposome.
18. A liposomal vaccine composition comprising:
a peptide antigen displayed on the surface of the liposome, which comprises, consists essentially of or consists of an amino acid sequence selected from G-I-L-E-D-M-P-V-D-P-D-N-E-A (SEQ ID NO: 1), L-E-D-M-P-V-D-P-D-N-E-A (SEQ ID NO: 38), and G-G-L-E-D-M-P-V-D-P-D-N-E-A (SEQ ID NO: 45); and
a peptide comprising a T-cell epitope which comprises, consists essentially of or consists of the amino acid sequence of SEQ ID NO: 32 (SAT47) or a close sequence analogue thereof, and
an adjuvant; wherein the adjuvant comprises a toll-like receptor 4 ligand; and
wherein the toll-like receptor 4 ligand comprises monophosphoryl lipid A (MPLA).
19. The liposomal vaccine composition of clause 18, wherein the peptide antigen comprises, consists essentially of or consists of the amino acid sequence G-I-L-E-D-M-P-V-D-P-D-N-E-A (SEQ ID NO: 1).
20. The liposomal vaccine composition of clause 18, wherein the peptide antigen comprises, consists essentially of or consists of the amino acid sequence L-E-D-M-P-V-D-P-D-N-E-A (SEQ ID NO: 38).
21. A pharmaceutical composition comprising the liposomal vaccine composition of any one of the clauses 1 to 20, and a pharmaceutically acceptable carrier, diluent and/or excipient.
22. A kit comprising a liposomal vaccine composition as defined in any one of clauses 1 to 20 or the pharmaceutical composition of clause 21 and a container.
23. The liposomal vaccine composition as defined in any one of clauses 1 to 20, or pharmaceutical composition as defined in clause 21, or kit as defined in clause 22 for use in the treatment or prevention of diseases, disorders or abnormalities associated with alpha-synuclein aggregates.
24. The liposomal vaccine composition, pharmaceutical composition or kit for use of clause 23, wherein the disease, disorder or abnormality associated with alpha-synuclein aggregates is a synucleinopathy.
25. The liposomal vaccine composition, pharmaceutical composition or kit for use of clause 23 or 24, wherein the disease, disorder or abnormality associated with alpha-synuclein aggregates or the synucleinopathy is selected from the group consisting of Parkinson's disease (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), diffuse Lewy body disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, and Down syndrome, multiple system atrophy (Shy-Drager syndrome, striatonigral degeneration and olivopontocerebellar atrophy), traumatic brain injury, chronic traumatic encephalopathy, dementia puglistica, tauopathies (Pick's disease, frontotemporal dementia, progressive supranuclear palsy, corticobasal degeneration and Niemann-Pick type C1 disease, frontotemporal dementia with Parkinsonism linked to chromosome 17), motor neuron disease, Huntington's disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Creutzfeldt-Jakob disease, ataxia telangiectatica, Meige's syndrome, subacute sclerosing panencephalitis, Gerstmann-Straussler-Scheinker disease, inclusion-body myositis, Gaucher disease, Krabbe disease as well as other lysosomal storage disorders (including Kufor-Rakeb syndrome and Sanfilippo syndrome) and rapid eye movement (REM) sleep behavior disorder.
26. The liposomal vaccine composition, pharmaceutical composition or kit for use according to any one of clauses 23 to 25, wherein the disease, disorder or abnormality associated with alpha-synuclein aggregates or the synucleinopathy is selected from the group consisting Lewy bodies and/or Lewy neurites and/or glial cytoplasmic inclusions, such as Parkinson's disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease.
27. The liposomal vaccine composition, pharmaceutical composition or kit for use according to any one of clauses 23 to 26, wherein the disease, disorder or abnormality associated with alpha-synuclein aggregates or the synucleinopathy is Multiple System Atrophy.
28. The liposomal vaccine composition, pharmaceutical composition or kit for use according to clauses 23 to 26, wherein the disease, disorder or abnormality associated with alpha-synuclein aggregates or the synucleinopathy is Parkinson's disease.
29. A method for prophylaxis, treatment and alleviation of diseases associated with the disease, disorder or abnormality associated with alpha-synuclein aggregates; wherein the method comprises administering to the subject the liposomal vaccine composition of any one of clauses 1 to 20 or the pharmaceutical composition of clause 21.
30. The method of clause 29 wherein the disease, disorder or abnormality associated with alpha-synuclein aggregates is a synucleinopathy.
31. The method of clause 29 or 30, wherein administering to the subject the liposomal vaccine composition of any one of clauses 1 to 20 or the pharmaceutical composition of clause 21 induces a protective immune response against alpha-synuclein aggregates.

In one aspect, the present invention relates to a liposomal composition comprising an antigenic peptide displayed on the surface of the liposome and an adjuvant, wherein the antigenic peptide has the structure:

X₁-X₂-X₃-E-X₄-X₅-P-V-D-P-D-N-E-X₆,
wherein:
E is glutamic acid, P is proline; V is Valine, D is aspartic acid, N is asparagine;
X₁, is present or not and, if present, is G, wherein G is glycine;
X₂, is present or not and, if present, is I or G, wherein I is isoleucine and G is defined as above;
X₃ is L, K, or S, wherein L is leucine, K is lysine, and S is serine;
X₄ is D, K or S, wherein D, K and S are as defined above;
X₅ is M, wherein M is methionine or methionine sulfoxide;
X₆ is A, K or S, wherein A is alanine and K and S are as defined above.

It is preferred that the peptide is modified as compared to this peptide sequence. Such peptides are able to elicit a strong anti-aSyn antibody response and the induced antibodies show high cross-reactivity with human aSyn even though these peptides have a sequence which is different from the native sequence. Immune responses superior to the response with the native sequence (i.e. targeting the same native structures) was achieved with the peptides according to the present invention. Thus, the invention relates to a liposomal vaccine composition comprising:
an antigenic peptide displayed on the surface of a liposome;
and an adjuvant;
   wherein the antigenic peptide comprises, consists essentially of, or consists of the structure:

X₁-X₂-X₃-E-X₄-X₅-P-V-D-P-D-N-E-X₆,
wherein:
E is glutamic acid, P is proline; V is Valine, D is aspartic acid, N is asparagine,
X₁, is present or not and, if present, is G, wherein G is glycine;
X₂, is present or not and, if present, is G, wherein G is defined as above;
X₃ is L, K, or S, wherein L is leucine, K is lysine, and S is serine;
X₄ is D, K or S, wherein D, K and S are as defined above;
X₅ is M, wherein M is methionine or methionine sulfoxide;
X₆ is A, K or S, wherein A is alanine and K, and S are as defined above;
with the proviso that X₃-E-X₄-X₅-P-V-D-P-D-N-E-X₆ is not L-E-D-M-P-V-D-P-D-N-E-A and wherein the antigenic peptide does not comprise the dipeptide Y-E immediately following X₆, wherein Y is tyrosine and E is as defined above. Thus, single letter amino acid code is generally used herein.

The antigenic peptide herein described may comprise between 1 and 5 amino acid differences (i.e. 1, 2, 3, 4 or 5 differences) or preferably between 1 and 4 amino acid differences compared with the amino acid sequence G-I-L-E-D-M-P-V-D-P-D-N-E-A. The differences are generally amino acid substitutions according to the options set out for each position. In some embodiments, however, X₆ is deleted. It is more preferred that there are 2, 3, 4 or 5 amino acid differences from the amino acid sequence G-I-L-E-D-M-P-V-D-P-D-N-E-A (SEQ ID NO: 1, which is the wild type alpha synuclein sequence from amino acids 111-124 of SEQ ID NO: 28). These differences can be selected from any of amino acids X₁-X₆. In some embodiments, the antigenic peptide comprises amino acid differences compared with the amino acid sequence G-I-L-E-D-M-P-V-D-P-D-N-E-A at one or more positions selected from X₁, X₂, X₃, X₄, X₅ and X₆. Absence of X₁, X₂ and/or X₆ is considered a difference.

Methionine is an amino acid that occurs naturally. The sulfur-containing amino acids methionine and cysteine are more easily oxidized than the other amino acids. Oxidation of the sulfur of methionine results in methionine sulfoxide or methionine sulfone. In some embodiments the antigenic peptides of the liposomal composition of the present invention comprise methionine in its oxidized form methionine sulfoxide. In some embodiments, a liposomal composition of the invention comprises an antigenic peptide comprising methionine sulfoxide. In an embodiment, X₅ is methionine or methionine sulfoxide. In an embodiment X₅ is methionine. In another embodiment, X₅ is methionine sulfoxide.

The liposomal composition of the invention comprises antigenic peptide that retain their ability to generate aSyn-specific antibodies when employed as an immunogen. Moreover, antigenic peptides of the invention are more immunogenic than the corresponding wild type aSyn peptide (comprising the 14-mer G-I-L-E-D-M-P-V-D-P-D-N-E-A, (SEQ ID NO: 1)) in terms of generating aSyn-specific antibodies, as demonstrated in the comparative experiments herein (example 2). They are also more immunogenic than other aSyn peptides from the C terminal region of aSyn, see Tables 2 and 4 below.

The antigenic peptides comprised into the liposomal vaccine composition of the invention typically do not comprise further alpha synuclein amino acid residues following X₆. In particular they do not comprise the dipeptide Y-E immediately following X₆. As described herein peptides including the amino acids Y₁₂₅ and E₁₂₆ are predicted by in silico analyses to bind with high affinity to different allelic variants of MHC I and thus represent potential aSyn specific cytotoxic T cell epitopes (www.syfpeithi.de). Thus, no amino acid extensions should (in contrast to the fusion proteins disclosed e.g. in WO 2005/108423 A1) be present C-terminally which represent the native amino acid sequence of aSyn, i.e. specifically Tyr₁₂₅ and Glu₁₂₆, which could bind with high affinity to different allelic variants of MHCI and thus could be potential cytotoxic T cell epitopes. Accordingly, no Y amino acid residue or YE dipeptide chain should be present at the C-terminal end of the peptide, wherein Y is tyrosine and E is as defined above.

The antigenic peptides included in the liposomal vaccine compositions may, however, comprise a limited number of further N terminal amino acid residues. The antigenic peptides may, additionally or alternatively, comprise a limited number of further C terminal amino acid residues in order to allow the insertion of the antigenic peptide into the liposome (in a manner that permits the peptide antigen to be displayed on the surface of the liposome). For example, the peptide can include additional residues, such as lysine residues to facilitate palmitoylation. Those residues are typically found at the N and/or C terminus of the peptide. In some embodiments, there may be 1-4 lysine residues added to the N and/or C terminus, preferably 2 lysine residues added at the N or C terminus, or preferably 2 lysine residues added at the N and C terminus. In this context, the term “consists essentially of” means that the alpha-synuclein-derived peptide antigen includes the 10 to 14 contiguous amino acids starting on position 111 or 113 of the alpha-synuclein (SEQ ID NO: 28) but can include a limited number of additional residues, such as two to four lysine residues to facilitate presentation on the surface of the liposome.

In some embodiments of the invention, one or both ends of the peptide antigen may include additional residues, which are not derived from the alpha-synuclein protein sequence but rather designed to modify the properties of the peptide. In some embodiments of the inventions, the peptide antigen of the invention further comprises a patch comprising, consisting essentially of or consisting of at least one amino acid, optionally arginine (R) or glutamic acid (E). In some embodiments of the inventions, the peptide antigen of the invention further comprises a patch comprising, consisting essentially of or consisting of at least three amino acids, optionally arginine (R) or glutamic acid (E). These sequences are referred to as patches. They may, for example, consist of sequences such as repeat arginine (R) sequences consisting of between 3 (RRR) and 8 (RRRRRRRR) arginines or repeat glutamic acid (E) sequences consisting of between 3 (EEE) and 8 (EEEEEEEE) glutamic acids.

The antigenic peptides of the invention are thus typically 11-22 amino acids in length, preferably 12-14 amino acids in length (i.e. 12, 13 or 14 amino acids in length). It is particularly preferred that the antigenic peptides are 12 or 14 amino acids in length. The antigenic peptides of the liposomal vaccine composition produce an antibody response in the absence of a T-cell response. Thus, the antigenic peptides of the invention themselves do not typically contain T-cell epitopes, in particular cytotoxic T-cell epitopes.

Liposomal vaccine compositions of the invention comprising antigenic peptides described herein are expected to be highly immunogenic and induced antibody responses are expected to have a low patient-to-patient variability. Furthermore, liposomal vaccine compositions of the invention incorporating palmitoylated peptides can also preferentially generate antibodies against alpha-synuclein sequences in a 3-sheet conformation, leading to an increased affinity for pathological and/or aggregated aSyn species. Periodic peptide arrangements, such as in the liposomal formulation, are known to crosslink the immunoglobulin antigen receptor on B cells in a very effective way providing a strong stimulus for antibody production (Pihlgren et al., 2013).

In one embodiment, a liposomal composition is provided comprising an antigenic peptide displayed on the surface of the liposome, wherein the antigenic peptide has the structure:

X₁-X₂-X₃-E-X₄-X₅-P-V-D-P-D-N-E-X₆,
wherein:
E is glutamic acid, P is proline; V is Valine, D is aspartic acid, N is asparagine;
X₁, is present or not and, if present, is G, wherein G is glycine;
X₂, is present or not and, if present, is I or G, wherein I is isoleucine and G is defined as above;
X₃ is L, K, or S, wherein L is leucine, K is lysine, and S is serine;
X₄ is D, K or S, wherein D, K and S are as defined above;
X₅ is M, wherein M is methionine or Methionine sulfoxide;
X₆ is A, K or S, wherein A is alanine and K, and S are as defined above.

In one embodiment, a liposomal composition is provided comprising an antigenic peptide displayed on the surface of the liposome, wherein the antigenic peptide has the structure:

X₁-X₂-X₃-E-X₄-X₅-P-V-D-P-D-N-E-X₆,
wherein:
E is glutamic acid, P is proline; V is Valine, D is aspartic acid, N is asparagine;
X₁, is present or not and, if present, is G wherein G is glycine;
X₂, is present or not and, if present, is G, wherein G is defined as above;
X₃ is L, K, or S, wherein L is leucine, K is lysine, and S is serine;
X₄ is D, K or S, wherein D, K and S are as defined above;
X₅ is M, wherein M is methionine or Methionine sulfoxide;
X₆ is A, K or S, wherein A is alanine and K, and S are as defined above;
with the proviso that X₃-E-X₄-M-P-V-D-P-D-N-E-X₆ is not L-E-D-M-P-V-D-P-D-N-E-A.

In some embodiments, X₁ and X₂ are absent. In certain embodiments, the present invention relates to a liposomal composition comprising an antigenic peptide having the structure:

X₃-E-X₄-X₅-P-V-D-P-D-N-E-X₆,
wherein:
E is glutamic acid, P is proline; V is Valine, D is aspartic acid, N is asparagine;
X₃ is L, K, or S, wherein L is leucine, K is lysine, and S is serine;
X₄ is D, K or S, wherein D, K and S are as defined above;
X₅ is M, wherein M is methionine, or methionine sulfoxide;
X₆ is A, K or S, wherein A is alanine and K, and S are as defined above;
with the proviso that X₃-E-X₄-X₅-P-V-D-P-D-N-E-X₆ is not L-E-D-M-P-V-D-P-D-N-E-A.

In some embodiments, X₁ is present and is G. In some embodiments, X₂ is present and is G. In some embodiments, if X₁ is present, X₂ is present and X₂ is G. In some embodiments X₃ is L, K, or S. In some embodiments X₄ is D, S or K. In some embodiments X₅ is Methionine or Methionine sulfoxide, preferably Methionine sulfoxide. In some embodiments, X₆ is A, K or S.

In some embodiments, X₁, X₂ are present and are each G, and X₃ is K.

In some embodiments, X₁, X₂ are present and are each G, X₃ is K, and X₆ is A.

In preferred embodiments, X₁, X₂ are present and are each G, X₃ is K, X₄ is S and X₆ is A. In preferred embodiments, X₁, X₂ are present and are each G, X₃ is K, X₄ is D and X₆ is A.

In some embodiments, X₃ is L or K.

In some embodiments, X₄ is D or S.

In some embodiments, X₅ is M.

In some embodiments, X₆ is A or S.

In some embodiments X₁ and X₂ are absent.

In some embodiments, X₁, X₂ are absent, and X₃ is L.

In preferred embodiments, X₁, X₂ are absent, X₃ is L, X₆ is S. In another preferred embodiment, X₁, X₂ are absent, X₃ is L and X₄ is D.

The peptide antigen included in the liposomal compositions of the invention may further comprise at least one chemical modification. Modifications, such as amidation, esterification, palmitoylation, formylation, acetylation, other chemical substitution etc. of a free C-terminal (or N-terminal) end of a peptide or its side chains are explicitly included within the scope of the invention. Such modifications are within the scope of the term “consisting essentially of” as used herein even if not separately specified. Palmityolation is a preferred modification to facilitate peptide antigen display on the surface of the liposome, which may be in a structural arrangement conducive to production of neutralizing antibodies.

The peptides according to the present invention (the “(antigenic) peptide(s) (of the present invention)”; “X₁ to X₆”; etc.) can be provided in compositions suitable for the intended use for preventing and/or treating synucleinopathies, especially in pharmaceutical compositions, preferably combined with a pharmaceutically acceptable carrier. Such pharmaceutical compositions can be administered to a patient in need thereof in an effective amount to achieve the preventive and/or therapeutic effect, as discussed in further detail herein.

In certain embodiments, the antigenic peptide according to the present invention is selected from the group consisting of GILEDMPVDPDNEA (SEQ ID NO: 1), GGKESMPVDPDNEA (SEQ ID NO: 2), GGKEDMPVDPDNEA (SEQ ID NO: 3), LESMPVDPDNES (SEQ ID NO: 4), LEKMPVDPDNEA (SEQ ID NO: 5), GGSESMPVDPDNEA (SEQ ID NO: 6), GGSESMPVDPDNES (SEQ ID NO: 7), KEDMPVDPDNEA (SEQ ID NO: 8), SESMPVDPDNEA (SEQ ID NO: 9), LESMPVDPDNEA (SEQ ID NO: 10), GGKESMPVDPDNES (SEQ ID NO: 11), KESMPVDPDNEA (SEQ ID NO: 12), KESMPVDPDNES (SEQ ID NO: 13), SEKMPVDPDNEA (SEQ ID NO: 14), LEKMPVDPDNES (SEQ ID NO: 15), GGSESMPVDPDNEK (SEQ ID NO: 16), LEKMPVDPDNEK (SEQ ID NO: 17), GGKEKMPVDPDNEA (SEQ ID NO: 18), GGKESMPVDPDNEK (SEQ ID NO: 19), GGKEKMPVDPDNEK (SEQ ID NO: 20), LEDMPVDPDNEA (SEQ ID NO: 38) and GGLEDMPVDPDNEA (SEQ ID NO: 45).

Thus, in certain embodiments, the antigenic peptide according to the present invention is selected from the group consisting of GGKESMPVDPDNEA (SEQ ID NO: 2), GGKEDMPVDPDNEA (SEQ ID NO: 3), LESMPVDPDNES (SEQ ID NO: 4), LEKMPVDPDNEA (SEQ ID NO: 5), GGSESMPVDPDNEA (SEQ ID NO: 6), GGSESMPVDPDNES (SEQ ID NO: 7), KEDMPVDPDNEA (SEQ ID NO: 8), SESMPVDPDNEA (SEQ ID NO: 9), LESMPVDPDNEA (SEQ ID NO: 10), GGKESMPVDPDNES (SEQ ID NO: 11), KESMPVDPDNEA (SEQ ID NO: 12), KESMPVDPDNES (SEQ ID NO: 13), SEKMPVDPDNEA (SEQ ID NO: 14), LEKMPVDPDNES (SEQ ID NO: 15), GGSESMPVDPDNEK (SEQ ID NO: 16), LEKMPVDPDNEK (SEQ ID NO: 17), GGKEKMPVDPDNEA (SEQ ID NO: 18), GGKESMPVDPDNEK (SEQ ID NO: 19) and GGKEKMPVDPDNEK (SEQ ID NO: 20).

Preferably, the antigenic peptide according to the present invention is selected from the group consisting of GGKESMPVDPDNEA (SEQ ID NO: 2), GGKEDMPVDPDNEA (SEQ ID NO: 3), LESMPVDPDNES (SEQ ID NO: 4), LEKMPVDPDNEA (SEQ ID NO: 5) and KESMPVDPDNEA (SEQ ID NO: 12).

Even more preferably the antigenic peptide according to the present invention is selected from the group consisting of GGKESMPVDPDNEA (SEQ ID NO: 2), GGKEDMPVDPDNEA (SEQ ID NO: 3), LESMPVDPDNES (SEQ ID NO: 4). In one embodiment, the antigenic peptide according to the present invention is GGKESMPVDPDNEA (SEQ ID NO: 2). In one embodiment, the antigenic peptide according to the present invention is KESMPVDPDNEA (SEQ ID NO: 12).

According to these embodiments, it is preferred that there is a maximum of one, two, three four or five mutations in the antigenic peptide compared with the native G-I-L-E-D-M-P-V-D-P-D-N-E-A sequence. In some embodiments there are no mutations in the antigenic peptide compared with the native G-I-L-E-D-M-P-V-D-P-D-N-E-A sequence. The mutations are amino acid substitutions according to the options set out for each position X₁ to X₆.

The alpha-synuclein-derived peptide antigen is displayed on the surface of the liposome. This is typically by insertion into the outer surface of the liposome. Insertion into the outer surface of the liposome may be facilitated through attachment of the alpha-synuclein-derived peptide antigen to a moiety that inserts into the outer surface of the liposome. The liposome may be any liposome that is suitable to present the alpha-synuclein-derived peptide antigen on the surface and also encapsulate a peptide comprising a T-cell epitope, in particular an aSyn irrelevant peptide comprising a universal T-helper cell epitope. Typically, the moiety comprises a hydrophobic moiety to ensure insertion into the lipid bilayer of a liposome. The moiety may be any suitable moiety but is preferably a fatty acid. The fatty acid may comprise a palmitoyl residue.

In one embodiment of the invention, the liposomal composition comprises an antigenic peptide described herein attached to one or two palmitoyl residues in the N or in the C terminal regions of the peptide. Thus, the antigenic peptide is mono- or di-palmitolyated on one and optionally both, of the terminal regions of the peptide. This may be facilitated by incorporating lysine residues in the N and C terminal regions of the alpha-synuclein-derived peptide antigen. The lysine residues are palmitoylated.

In another embodiment of the invention, the liposomal composition comprises an antigenic peptide described herein attached to at least one palmitoyl residue in the N and C terminal regions of the antigenic peptide. Thus, the antigenic peptide is dipalmitolyated. This may be facilitated by incorporating at least one lysine residue in the N and C terminal regions of the alpha-synuclein-derived peptide antigen. The lysine residues are palmitoylated.

In another embodiment of the invention, the liposomal composition comprises the antigenic peptide attached to two palmitoyl residues in the N and C terminal regions of the peptide. Thus, the antigenic peptide is tetrapalmitolyated. This may be facilitated by incorporating two lysine residues in the N and C terminal regions of the alpha-synuclein-derived peptide antigen. The lysine residues are palmitoylated.

The liposomal vaccine compositions of the invention comprise a peptide comprising a T-cell epitope, in particular a T-helper cell epitope. Preferably, the peptide comprising a T-cell epitope comprises at least two T-cell epitopes. The peptide comprising a T-cell epitope may be at least partially encapsulated in the liposome and/or may be also presented on the surface of the liposome and/or may be at least partially incorporated into the lipid bilayer of the liposome. It is preferred that the peptide comprising a T-cell epitope is encapsulated. This is typically achieved by forming the liposome in a solution containing the peptide comprising a T-cell epitope. It should be noted that during the encapsulation process a proportion of the peptide comprising a T-cell epitope may adsorb to the outer surface of the liposome and a proportion of the peptide comprising a T-cell epitope may become incorporated into the lipid bilayer of the liposome. Thus, when reference is made to an encapsulated T-cell epitope it is intended to mean that at least some of the peptide comprising a T-cell epitope is encapsulated.

As a proportion of the total peptide comprising a T-cell epitope which is associated with the liposome, this may represent at least 20%, 25%, 30%, 35%, 40%, 50%, 60%, 70%, 80% or 90% (with the remainder adsorbed to the surface and incorporated in the lipid bilayer). The precise degree of encapsulation will depend upon the properties of the peptide comprising a T-cell epitope.

In some embodiments of the invention, the liposomal vaccine composition comprises at least one or at least two (2) T-cell epitopes. The T-cell epitopes can be at least two (2) linear T-cell epitopes, or a conjugate of several T-cell epitopes linked together with or without a spacer. In some embodiment of the invention, the T-cell epitopes comprises several T-cell epitopes linked together with a spacer comprising at least one amino acid, preferably at least three amino acids, more preferably an amino acid sequence comprising VVR. The conjugate comprises at least two, three or four different T-cell epitopes encapsulated within the liposome. In some embodiments, the T-cell epitopes are derived from diphtheria toxin, tetanus toxin, Epstein Barr Virus, influenza hemagglutinin and/or keyhole limpet hemocyanin. Specific preferred combinations of T-cell epitopes are therefore selected from:
a) A combination of a diphtheria toxin and tetanus toxin T-cell epitope
b) A combination of an Epstein Barr Virus and tetanus toxin T-cell epitope
c) A combination of an Epstein Barr Virus, tetanus toxin and keyhole limpet hemocyanin T-cell epitope; or
d) A combination of an influenza hemagglutinin, diphtheria toxin, tetanus toxin and Epstein Barr Virus T-cell epitope.

Whilst the above combinations are preferably included in the order specified, they may be included in an alternative order. For example, if there are three T-cell epitopes, A, B and C, they may be included in any of orders ABC, ACB, BAC, BCA, CAB or CBA.

Such peptides are preferably included in the vaccine compositions of the invention. Thus, peptides useful in the invention comprise, consist essentially of or consist of an amino acid sequence selected from SEQ ID NO: 21 (SAT13), SEQ ID NO: 22 (SAT15), SEQ ID NO: 23 (SAT17), SEQ ID NO: 24, SEQ ID NO: 25, SEQ ID NO: 26, SEQ ID NO: 27 and/or any combination thereof. In a preferred embodiment, the T-cell epitope comprises, consists essentially of or consists of an amino acid sequence selected from SEQ ID NO: 24, SEQ ID NO: 25, SEQ ID NO: 26, SEQ ID NO: 27 and/or any combination thereof. In some embodiments, the T-cell epitope comprises, consists essentially of or consists of SEQ ID NO: 24. In some embodiments, the T-cell epitope comprises, consists essentially of or consists of SEQ ID NO: 25. In some embodiments, the T-cell epitope comprises, consists essentially of or consists of SEQ ID NO: 26. In some embodiments, the T-cell epitope comprises, consists essentially of or consists of SEQ ID NO: 27. In other embodiments, the T-cell epitope comprises at least two amino acid sequences selected from SEQ ID NO: 24, SEQ ID NO: 25, SEQ ID NO: 26 and SEQ ID NO: 27. In another embodiments, the T-cell epitope comprises at least three amino acid sequences selected from SEQ ID NO: 24, SEQ ID NO: 25, SEQ ID NO: 26 and SEQ ID NO: 27. In a most preferred embodiment the T-cell epitope comprises SEQ ID NO: 24, SEQ ID NO: 25, SEQ ID NO: 26 and SEQ ID NO: 27. Combinations of these peptides, trimmed to 10-20 amino acids in length as appropriate, can also be included in the vaccine compositions of the invention. The combined peptides are preferably joined by one or more linkers as defined herein.

In some embodiments, the T-cell epitopes of the invention may include one or more modifications, such as, but not limited to amino acid substitutions and post-translational modification. In a more preferred embodiment, the T-cell epitopes of the inventions include one to five amino acid modifications.

Pan DR epitope (PADRE or PaDre) peptides (having sequence AKFVAAWTLKAAA (SEQ ID NO: 35) are known, see, e.g., Alexander et al. (1994), and de Guercio et al. (1997). It has been found that PADRE peptides are peptides that deliver help for antibody responses and provide helper T-cell activity in vivo. These properties suggest that constructs containing the PADRE peptides might be as efficient at generating an immune response as large multivalent antigens.

P2 (having the amino acid sequence: QYIKANSKFIGITEL (SEQ ID NO: 36)) and P30 peptides (having the amino acid sequence: FNNFTVSFWLRVPKVSASHLE (SEQ ID NO: 37)) were derived from tetanus toxin, see, e.g., Panina-Bordignon et al (1989); and Boeckler et al., (1999).

In some embodiments, the liposomal vaccine composition of the invention comprises a universal T-cell epitope comprising, consisting essentially of or consisting of an amino acid sequence selected from SEQ ID NO: 29 (SAT42), SEQ ID NO: 32 (SAT47) or a close sequence analogue as defined herein. In some embodiments, the liposomal vaccine composition of the invention comprises a universal T-cell epitope comprising, consisting essentially of or consisting of SEQ ID NO: 32 (SAT47) or a close sequence analogue as defined herein.

In some embodiments, the liposomal vaccine composition of the invention comprises a peptide having a universal T-cell epitope comprising, consisting essentially of, or consisting of SEQ ID NO: 32 (SAT47) or SEQ ID NO: 33 (SAT58). In some embodiments, the liposomal vaccine composition of the invention comprises a peptide having a universal T-cell epitope comprising, consisting essentially of, or consisting of SEQ ID NO: 33 (SAT58).

Preferably, the combination of T-cell epitopes are provided in a peptide comprising the amino acid sequence of SEQ ID NO: 29 (SAT42), SEQ ID NO: 30 (SAT43), SEQ ID NO: 31 (SAT44), SEQ ID NO: 32 (SAT47), SEQ ID NO: 35 (PaDre), SEQ ID NO: 36 (P2), SEQ ID NO: 37 (P30), SEQ ID NO: 21 (SAT 13), SEQ ID NO: 22 (SAT 15), SEQ ID NO: 23 (SAT17), SEQ ID NO: 24, SEQ ID NO: 25, SEQ ID NO: 26 or SEQ ID NO: 27, or a variant (which may be referred to as a close sequence analogue) having at least 75%, 80%, 85%, 90%, 95%, 96%, 97%, 98%, or preferably 99% sequence identity therewith or a variant comprising between one and five amino acid substitutions, with the proviso that the resultant peptide retains a T-cell epitope.

In one embodiment, the combination of T-cell epitopes are provided in a peptide comprising the amino acid sequence of SEQ ID NO: 32 (SAT47), or a variant (which may be referred to as a close sequence analogue) having at least 75%, 80%, 85%, 90%, 95%, 96%, 97%, 98%, or preferably 99% sequence identity therewith or a variant comprising between one and five amino acid substitutions, with the proviso that the resultant peptide retains a T-cell epitope.

In one embodiment, the combination of T-cell epitopes are provided in a peptide comprising the amino acid sequence of SEQ ID NO: 33 (SAT58), or a variant (which may be referred to as a close sequence analogue) having at least 75%, 80%, 85%, 90%, 95%, 96%, 97%, 98%, or preferably 99% sequence identity therewith or a variant comprising between one and five amino acid substitutions, with the proviso that the resultant peptide retains a T-cell epitope.

Alignment of a variant is typically performed based on comparison with the full length sequence of the original linear peptide. Various alignment algorithms are available and in routine use, such as CLUSTALW and GAP. The composition of these peptides is explained in more detail with reference to Table 1 below. Such peptides are included in the liposomal vaccine compositions of the invention. Preferably, the liposomal vaccine composition of the invention comprises a peptide comprising the amino acid sequence of SEQ ID NO: 29 (SAT42), SEQ ID NO: 32 (SAT47) or a close sequence analogue thereof as defined herein. Even more preferably, the liposomal vaccine composition comprises a peptide comprising the amino acid sequence of SEQ ID NO: 32 (SAT 47) or a close sequence analogue thereof. For example, the liposomal vaccine composition may comprise a peptide comprising the amino acid sequence of SEQ ID NO 33 (SAT58).

TABLE 1

| Name | Sequence | Peptide origin |
|---|---|---|
| SAT42 | VHHNTEEIVAQSIALSSLMVPMGAPQYIKAN | Diphtheria Toxin + |
| | SKFIGITEL | Tetanus toxin |
| | (SEQ ID NO: 29) | |
| SAT43 | VYGGSKTSLYNLRRGTALAIVVRQYIKANSK | Epstein Barr + |
| | FIGITELVVRPIFFLHHSNTDRLWAI | Tetanus + KLH |
| | (SEQ ID NO: 30) | |
| SAT44 | VYGGSKTSLYNLRRGTALAIVVRQYIKANSK | Epstein Barr + |
| | FIGITEL | Tetanus |
| | (SEQ ID NO: 31) | |
| SAT47 | SMGVYQILAIYSTVVRIVAQSIALSSVVRYI | Influenza hemagglutinin + |
| | KANSKFIGVVRLYNLRRGTAL | Difteria + Tetanus + |
| | (SEQ ID NO: 32) | Epstein Barr |
| SAT58 | SAGVYQILAIYSTVVRIVAQSIALSSVVRYI | Influenza hemagglutinin + |
| | KANSKFIGVVRLYNLRRGTAL | Difteria + Tetanus + |
| | (SEQ ID NO: 33) | Epstein Barr |
| SAT13 | VHHNTEEIVAQSIALSSLMV | Diphtheria Toxin |
| | SEQ ID NO: 21 | |
| SAT15 | IDGVKLESMGVYQILAIYSTVASSL | Influenza hemagglutinin |
| | SEQ ID NO: 22 | |
| SAT17 | VYGGSKTSLYNLRRGTALAI | Epstein Barr Virus |
| | SEQ ID NO: 23 | |
| Trimmed | SMGVYQILAIYST | Influenza hemagglutinin |
| peptide 1 | SEQ ID No: 24 | |
| Trimmed | IVAQSIALSS | Diphtheria Toxin |
| peptide 2 | SEQ ID NO: 25 | |
| Trimmed P2 | YIKANSKFIG | Tetanus toxin |
| | SEQ ID No: 26 | |
| Trimmed | LYNLRRGTAL | Epstein Barr Virus |
| peptide 3 | SEQ ID NO: 27 | |
| Trimmed | SAGVYQILAIYST | Influenza hemagglutinin |
| peptide 4 | SEQ ID NO: 46 | |

In some embodiments of the invention, the T-cell epitopes may be encapsulated or displayed on the surface of the liposome. Depending on the nature of the T-cell epitope, it may to some extent bind or associate with the liposome membrane enabling some level of surface display and/or some level within the liposomal membrane.

In some embodiments, the liposome has a negative surface charge; the liposome is anionic. Preferably, the liposome comprises phospholipids and even more preferably, the phospholipids comprise dimyrsitoylphosphatidyl-choline (DMPC) and dimyrsitoylphosphatidyl-glycerol (DMPG). The liposome may further comprise cholesterol. The molar ratios of these three components may be 9:1:7 in some embodiments.

A most preferred construction therefore comprises the antigenic peptide reconstituted in the liposome. Accordingly, these compositions of the invention may generally be referred to herein as “liposomal vaccine compositions of the invention”.

The compositions of the invention typically comprise at least one adjuvant. In some embodiments of the invention, the compositions of the invention comprise two adjuvants. The purpose of the adjuvant(s) is to increase or stimulate the immune response in the subject. Preferably, the at least one adjuvant is part of the carrier (as opposed to being encapsulated within the carrier). Thus, the at least one adjuvant may form part of a liposome; it may form part of the lipid bilayer. The adjuvant may be a TLR agonist. The adjuvant may therefore be a lipid-based adjuvant. The adjuvant may be, at least in part, displayed on the surface of the liposome; this may be as a consequence of the adjuvant forming part of the lipid bilayer. The adjuvant may comprise a TLR4 agonist and/or a TLR9 agonist.

Examples of TLR4 ligands useful for the invention include a TLR4 agonist, including, but not limited to, monophosphoryl lipid A (MPLA). As used herein, the term “monophosphoryl lipid A” or MPLA” refers to a modified form of lipid A, which is the biologically active part of Gram-negative bacterial lipopolysaccharide (LPS) endotoxin. MPLA is less toxic than LPS while maintaining the immunostimulatory activity. As a vaccine adjuvant, MPLA stimulates both cellular and humoral responses to the vaccine antigen. Examples of MPLA include, but are not limited to, 3-O-desacyl-4′-monophosphoryl lipid A, monophosphoryl hexa-acyl lipid A, 3-deacyl, monophosphoryl 3-deacyl lipid A, and structurally related variants thereof. MPLA useful for the invention can be obtained using methods known in the art, or from a commercial source, such as 3D-(6-acyl) PHAD®, PHAD®, PHAD®-504, 3D-PHAD® from Avanti Polar Lipids (Alabaster, Alabama, USA) or MPL™ from various commercial sources. According to particular embodiments, the TLR4 agonist is MPLA.

Examples of TLR9 ligands useful for the invention include a TLR9 agonist including, but not limited to, CpG oligonucleotides.

Other adjuvants that may be employed according to the invention include aluminium hydroxide (Alum) and/or CpG amongst others. Any suitable CpG known to those skilled in the art can be used in the invention in view of the present disclosure. Examples of such CpG oligonucleotides include, but are not limited to CpG2006 (also known as CpG 7909), CpG 1018, CpG2395, CpG2216 or CpG2336. A CpG can be lipidated using methods known in the art in view of the present disclosure. In some embodiments, 3′ terminus or 5′ terminus of a CpG oligonucleotide is covalently linked to a cholesterol molecule through a phosphate bond, optionally via a PEG linker. Thus, a preferred adjuvant is CpG-chol (cholesterol). In some embodiments of the invention the CpG comprises CpG 2006 (also known as CpG 7909) as defined by the following nucleotide sequence (SEQ ID NO: 34):

(SEQ ID NO: 34)
5′-tcgtcgttttgtcgttttgtcgtt-3′

wherein bases are phosphorothioate (ps).

In some embodiments of the invention, a liposomal vaccine composition comprises:
a. A peptide antigen displayed on the surface of the liposome, which comprises, consists essentially of or consists of an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20;
b. A peptide comprising a T-cell epitope; and
c. An adjuvant.

In some embodiments of the invention, the liposomal vaccine composition comprises:
a. A peptide antigen displayed on the surface of the liposome, which comprises, consists essentially of or consists of an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20;
b. A peptide comprising a T-cell epitope; and
c. An adjuvant,
wherein the adjuvant comprises at least one TLR ligand.

In some embodiments of the invention, the liposomal vaccine composition comprises:
a. A peptide antigen displayed on the surface of the liposome, which comprises, consists essentially of or consists of an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20;
b. A peptide comprising a T-cell epitope; and
c. An adjuvant,
   wherein the adjuvant comprises at least one of a TLR4 ligand and/or a TLR9 ligand.

In some embodiments of the invention, the liposomal vaccine composition comprises:
a. A peptide antigen displayed on the surface of the liposome, which comprises, consists essentially of or consists of an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20;
b. A peptide comprising a T-cell epitope which comprises, consists essentially of or consists of an amino acid sequence selected from the group consisting of SEQ ID NO: 24, SEQ ID NO: 25, SEQ ID NO: 26, and SEQ ID NO: 27 or any combination thereof; and
c. An adjuvant,
   wherein the adjuvant comprises at least one of a TLR4 ligand and/or a TLR9 ligand.

In some embodiments of the invention, the liposomal vaccine composition comprises:
a. A peptide antigen displayed on the surface of the liposome, which comprises, consists essentially of or consists of an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 10, SEQ ID NO:11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20;
b. A peptide comprising a T-cell epitope which comprises, consists essentially of or consists of SEQ ID NO: 32 (SAT47) or a close sequence analogue of SEQ ID NO: 32; and
c. An adjuvant,
   wherein the adjuvant comprises at least one of a TLR4 ligand and/or a TLR9 ligand.

In some embodiments of the invention the liposomal vaccine composition comprises:
a. a peptide antigen displayed on the surface of the liposome, which comprises, consists essentially of or consists of an amino acid sequence selected from SEQ ID NO: 2, SEQ ID NO 3, SEQ ID NO 4, SEQ ID NO 5 or SEQ ID NO:12,
b. a peptide comprising a T-cell epitope which comprises, consists essentially of, or consists of the amino acid sequence of SEQ ID NO: 24 and/or SEQ ID NO: 25 and/or SEQ ID NO: 27 and
c. an adjuvant; wherein the adjuvant comprises a toll-like receptor 4 ligand, preferably monophosphoryl lipid A (MPLA), and/or a toll-like receptor 9 ligand, preferably CpG.

In some embodiments of the invention the liposomal vaccine composition comprises:
a. a peptide antigen displayed on the surface of the liposome, which comprises, consists essentially of or consists of an amino acid sequence selected from SEQ ID NO: 2, SEQ ID NO 3, SEQ ID NO 4, SEQ ID NO 5 or SEQ ID NO:12,
b. a peptide comprising a T-cell epitope which comprises, consists essentially of, or consists of the amino acid sequence of SEQ ID NO: 24, and SEQ ID NO: 25, and SEQ ID NO: 27 and
c. an adjuvant; wherein the adjuvant comprises a toll-like receptor 4 ligand, preferably monophosphoryl lipid A (MPLA), and/or a toll-like receptor 9 ligand, preferably CpG.

In some embodiments of the invention the liposomal vaccine composition comprises:
a. a peptide antigen displayed on the surface of the liposome, which comprises, consists essentially of or consists of an amino acid sequence selected from SEQ ID NO: 2, SEQ ID NO 3, SEQ ID NO 4, SEQ ID NO 5 or SEQ ID NO:12,
b. a peptide comprising a T-cell epitope which comprises, consists essentially of, or consists of the amino acid sequence of SEQ ID NO: 46 and/or SEQ ID NO: 25 and/or SEQ ID NO: 27 and
c. an adjuvant; wherein the adjuvant comprises a toll-like receptor 4 ligand, preferably monophosphoryl lipid A (MPLA), and/or a toll-like receptor 9 ligand, preferably CpG.

In some embodiments of the invention the liposomal vaccine composition comprises:
a. a peptide antigen displayed on the surface of the liposome, which comprises, consists essentially of or consists of an amino acid sequence selected from SEQ ID NO: 2, SEQ ID NO 3, SEQ ID NO 4, SEQ ID NO 5 or SEQ ID NO:12,
b. a peptide comprising a T-cell epitope which comprises, consists essentially of, or consists of the amino acid sequence of SEQ ID NO: 46, and SEQ ID NO: 25, and SEQ ID NO: 27 and
c. an adjuvant; wherein the adjuvant comprises a toll-like receptor 4 ligand, preferably monophosphoryl lipid A (MPLA), and/or a toll-like receptor 9 ligand, preferably CpG.

In some embodiments of the invention the liposomal vaccine composition comprises:
a. a peptide antigen displayed on the surface of the liposome, which comprises, consists essentially of or consists of an amino acid sequence SEQ ID NO: 2, or SEQ ID NO:12,
b. a peptide comprising a T-cell epitope which comprises, consists essentially of, or consists of the amino acid sequence of SEQ ID NO: 46, and/or SEQ ID NO: 25, and/or SEQ ID NO: 27 and
c. an adjuvant; wherein the adjuvant comprises a toll-like receptor 4 ligand, preferably monophosphoryl lipid A (MPLA), and/or a toll-like receptor 9 ligand, preferably CpG.

In some embodiments of the invention the liposomal vaccine composition comprises:
a. a peptide antigen displayed on the surface of the liposome, which comprises, consists essentially of or consists of an amino acid sequence SEQ ID NO: 2, or SEQ ID NO:12,
b. a peptide comprising a T-cell epitope which comprises, consists essentially of, or consists of the amino acid sequence of SEQ ID NO: 46, and SEQ ID NO: 25, and SEQ ID NO: 27 and
c. an adjuvant; wherein the adjuvant comprises a toll-like receptor 4 ligand, preferably monophosphoryl lipid A (MPLA), and/or a toll-like receptor 9 ligand, preferably CpG.

In some embodiments of the invention, the liposomal vaccine composition comprises:
a. a peptide antigen displayed on the surface of the liposome, which comprises, consists essentially of or consists of an amino acid sequence selected from the group consisting of SEQ ID NO: 2, SEQ ID NO: 3, and SEQ ID NO: 4;
b. a peptide comprising a T-cell epitope; and
c. an adjuvant.

In some embodiments of the invention, the liposomal vaccine composition comprises:
a. a peptide antigen displayed on the surface of the liposome, which comprises, consists essentially of or consists of an amino acid sequence selected from the group consisting of SEQ ID NO: 2, SEQ ID NO: 3, SEQ ID NO: 4 and SEQ ID NO: 12;
b. a peptide comprising a T-cell epitope; and
c. an adjuvant.

In further embodiments of the invention, the liposomal vaccine composition comprises:
a. a peptide antigen displayed on the surface of the liposome, which comprises, consists essentially of or consists of an amino acid sequence selected from the group consisting of SEQ ID NO: 2, SEQ ID NO: 3, and SEQ ID NO: 4;
b. a peptide comprising a T-cell epitope which comprises, consists essentially of or consists of an amino acid sequence selected from the group consisting of SEQ ID NO: 24, SEQ ID NO: 25, SEQ ID NO: 26, and SEQ ID NO: 27 or any combination thereof; and
c. an adjuvant.

In further embodiments of the invention, the liposomal vaccine composition comprises:
a. a peptide antigen displayed on the surface of the liposome, which comprises, consists essentially of or consists of an amino acid sequence selected from the group consisting of SEQ ID NO: 2, SEQ ID NO: 3, SEQ ID NO: 4 and SEQ ID NO: 12;
b. a peptide comprising a T-cell epitope which comprises, consists essentially of or consists of an amino acid sequences SEQ ID NO: 24 and SEQ ID NO: 25 and SEQ ID NO: 26, and SEQ ID NO: 27 or any combination thereof, and
c. an adjuvant.

In further embodiments of the invention, the liposomal vaccine composition comprises:
a. a peptide antigen displayed on the surface of the liposome, which comprises, consists essentially of or consists of an amino acid sequence selected from the group consisting of SEQ ID NO: 2, SEQ ID NO: 3, and SEQ ID NO: 4;
b. a peptide comprising a T-cell epitope which comprises, consists essentially of or consists of an amino acid sequence selected from the group consisting of SEQ ID NO: 46, SEQ ID NO: 25, SEQ ID NO: 26, and SEQ ID NO: 27, and
c. an adjuvant.

In further embodiments of the invention, the liposomal vaccine composition comprises:
a. a peptide antigen displayed on the surface of the liposome, which comprises, consists essentially of or consists of an amino acid sequence selected from the group consisting of SEQ ID NO: 2, SEQ ID NO: 3, SEQ ID NO: 4 and SEQ ID NO: 12;
b. a peptide comprising a T-cell epitope which comprises, consists essentially of or consists of an amino acid sequence SEQ ID NO: 46 and SEQ ID NO: 25 and SEQ ID NO: 26, and SEQ ID NO: 27; and
c. an adjuvant.

In some embodiments of the invention the liposomal vaccine composition comprises:
a. a peptide antigen displayed on the surface of the liposome, which comprises, consists essentially of or consists of an amino acid sequence SEQ ID NO: 2,
b. a peptide comprising a T-cell epitope which comprises, consists essentially of or consists of the amino acid sequence of SEQ ID NO: 32 (SAT47) or a close sequence analogue of SEQ ID NO: 32 and
c. an adjuvant.

In some embodiments the liposomal vaccine composition comprises:
a. a peptide antigen displayed on the surface of the liposome, which comprises, consists essentially of or consists of an amino acid sequence SEQ ID NO: 2,
b. a peptide comprising a T-cell epitope which comprises, consists essentially of or consists of the amino acid sequence of SEQ ID NO: 33 (SAT58) and
c. an adjuvant.

In some embodiments of the invention the liposomal vaccine composition comprises:
a. a peptide antigen displayed on the surface of the liposome, which comprises, consists essentially of or consists of an amino acid sequence SEQ ID NO: 12,
b. a peptide comprising a T-cell epitope which comprises, consists essentially of or consists of the amino acid sequence of SEQ ID NO: 32 (SAT47) or a close sequence analogue of SEQ ID NO: 32 and
c. an adjuvant.

In some embodiments of the invention the liposomal vaccine composition comprises:
a. a peptide antigen displayed on the surface of the liposome, which comprises, consists essentially of or consists of an amino acid sequence SEQ ID NO: 12,
b. a peptide comprising a T-cell epitope which comprises, consists essentially of or consists of the amino acid sequence of SEQ ID NO: 33 (SAT58) and
c. an adjuvant.

In some embodiment of the invention, the liposomal vaccine composition comprises:
a. a peptide antigen displayed on the surface of the liposome, which comprises, consists essentially of or consists of an amino acid sequence SEQ ID NO: 2,
b. a peptide comprising a T-cell epitope which comprises, consists essentially of or consists of the amino acid sequence of SEQ ID NO: 33 (SAT58); and
c. an adjuvant; wherein the adjuvant comprises a toll-like receptor 4 ligand and/or a toll-like receptor 9 ligand. Optionally the toll-like receptor 4 ligand comprises monophosphoryl lipid A (MPLA) and the toll-like receptor 9 ligand comprises CpG.

In some embodiments of the invention, a liposomal vaccine composition comprises:
a) a peptide antigen displayed on the surface of the liposome, which comprises, consists essentially of or consists of SEQ ID NO: 2, SEQ ID NO: 3, or SEQ ID NO: 4;
b) a peptide comprising a T-cell epitope, which comprises, consists essentially of or consists of SEQ ID NO: 32 (SAT47) or a close sequence analogue of SEQ ID NO: 32;
c) a TLR4 ligand comprising monophosphoryl lipid A (MPLA); and
d) a TLR9 ligand comprising CpG.

In some embodiments of the invention, a liposomal vaccine composition comprises:
a) a peptide antigen displayed on the surface of the liposome, which comprises, consists essentially of or consists of SEQ ID NO: 2, SEQ ID NO: 3, SEQ ID NO: 4 or SEQ ID NO: 12;
b) a peptide comprising a T-cell epitope, which comprises, consists essentially of or consists of SEQ ID NO: 32 (SAT47) or a close sequence analogue of SEQ ID NO: 32;
c) a TLR4 ligand comprising monophosphoryl lipid A (MPLA); and
d) a TLR9 ligand comprising CpG.

In some embodiments of the invention, a liposomal vaccine composition comprises:
a) a peptide antigen displayed on the surface of the liposome, which comprises, consists essentially of or consists of an amino acid sequence selected from SEQ ID NO: 2, SEQ ID NO: 3, SEQ ID No: 4, SEQ ID NO: 5 or SEQ ID NO: 12; and
b) a peptide comprising a T-cell epitope which comprises, consists essentially of or consists of the amino acid sequence of SEQ ID NO: 32 (SAT47) or a close sequence analogue thereof; and
c) an adjuvant; wherein the adjuvant comprises a toll-like receptor 4 ligand; and wherein the toll-like receptor 4 ligand comprises monophosphoryl lipid A (MPLA).

In some embodiments of the invention, a liposomal vaccine composition comprises:
a) a peptide antigen displayed on the surface of the liposome, which comprises, consists essentially of or consists of an amino acid sequence SEQ ID NO: 2,
b) a peptide comprising a T-cell epitope which comprises, consists essentially of or consists of the amino acid sequence of SEQ ID NO: 32 (SAT47) or a close sequence analogue thereof; and
c) an adjuvant; wherein the adjuvant comprises a toll-like receptor 4 ligand and/or a toll-like receptor 9 ligand; and wherein the toll-like receptor 4 ligand comprises monophosphoryl lipid A (MPLA), wherein the toll-like receptor 9 ligand comprises CpG.

In some embodiments of the invention, a liposomal vaccine composition comprises:
a) a peptide antigen displayed on the surface of the liposome, which comprises, consists essentially of or consists of SEQ ID NO: 2;
b) a peptide comprising a T-cell epitope, which comprises, consists essentially of or consists of SEQ ID NO: 32 (SAT47) or a close sequence analogue of SEQ ID NO: 32;
c) a TLR4 ligand comprising monophosphoryl lipid A (MPLA); and
d) a TLR9 ligand comprising CpG.

In some embodiments of the invention, a liposomal vaccine composition comprises:
a) a peptide antigen displayed on the surface of the liposome, which comprises, consists essentially of or consists of SEQ ID NO: 2;
b) a peptide comprising a T-cell epitope, which comprises, consists essentially of or consists of SEQ ID NO: 33 (SAT58);
c) a TLR4 ligand comprising monophosphoryl lipid A (MPLA); and
d) a TLR9 ligand comprising CpG.

In some embodiments of the invention, a liposomal vaccine composition comprising:
a) a peptide antigen displayed on the surface of the liposome, which comprises, consists essentially of or consists of an amino acid sequence selected from SEQ ID NO: 2, SEQ ID NO: 3, SEQ ID No: 4, SEQ ID NO: 5 or SEQ ID NO: 12; and
b) a peptide comprising a T-cell epitope which comprises, consists essentially of or consists of the amino acid sequence of SEQ ID NO: 33 (SAT58); and
c) an adjuvant; wherein the adjuvant comprises a toll-like receptor 4 ligand; and wherein the toll-like receptor 4 ligand comprises monophosphoryl lipid A (MPLA).

In some embodiments of the invention, a liposomal vaccine composition comprises:
a) a peptide antigen displayed on the surface of the liposome, which comprises, consists essentially of or consists of SEQ ID NO: 3;
b) a peptide comprising a T-cell epitope, which comprises, consists essentially of or consists of SEQ ID NO: 32 (SAT47) or a close sequence analogue of SEQ ID NO: 32;
c) a TLR4 ligand comprising monophosphoryl lipid A (MPLA); and
d) a TLR9 ligand comprising CpG.

In some embodiments of the invention, a liposomal vaccine composition comprises:
a) a peptide antigen displayed on the surface of the liposome, which comprises, consists essentially of or consists of SEQ ID NO: 4;
b) a peptide comprising a T-cell epitope, which comprises, consists essentially of or consists of SEQ ID NO: 32 (SAT47) or a close sequence analogue of SEQ ID NO: 32;
c) a TLR4 ligand comprising monophosphoryl lipid A (MPLA); and
d) a TLR9 ligand comprising CpG.

In some embodiments of the invention, a liposomal vaccine composition comprises:
a) a peptide antigen displayed on the surface of the liposome, which comprises, consists essentially of or consists of SEQ ID NO: 12;
b) a peptide comprising a T-cell epitope, which comprises, consists essentially of or consists of SEQ ID NO: 32 (SAT47) or a close sequence analogue of SEQ ID NO: 32;
c) a TLR4 ligand comprising monophosphoryl lipid A (MPLA); and
d) a TLR9 ligand comprising CpG.

In some embodiments of the invention, a liposomal vaccine composition comprises:
a) a peptide antigen displayed on the surface of the liposome, which comprises, consists essentially of or consists of SEQ ID NO: 2, SEQ ID NO: 3, or SEQ ID NO: 4;
b) a peptide comprising a T-cell epitope; and
c) an adjuvant,
wherein the peptide antigen comprises one or two palmitoyl residues in the N and/or in the C terminal regions of the peptide.

In some embodiments of the invention, a liposomal vaccine composition comprises:
a) a peptide antigen displayed on the surface of the liposome, which comprises, consists essentially of or consists of SEQ ID NO: 2, SEQ ID NO: 3, SEQ ID NO: 4 or SEQ ID NO: 12;
b) a peptide comprising a T-cell epitope; and
c) an adjuvant,
wherein the peptide antigen comprises one or two palmitoyl residues in the N and/or in the C terminal regions of the peptide.

In some embodiments of the invention, a liposomal vaccine composition comprises:
d) a peptide antigen displayed on the surface of the liposome, which comprises, consists essentially of or consists of SEQ ID NO: 2, SEQ ID NO: 3, or SEQ ID NO: 4;
e) a peptide comprising a T-cell epitope; and
f) an adjuvant,
wherein the peptide antigen comprises two palmitoyl residues in the N or in the C terminal regions of the peptide.

In some embodiments of the invention, a liposomal vaccine composition comprises:
a) a peptide antigen displayed on the surface of the liposome, which comprises, consists essentially of or consists of SEQ ID NO: 2, SEQ ID NO: 3, or SEQ ID NO: 4;
b) a peptide comprising a T-cell epitope; and
c) an adjuvant,
wherein the peptide antigen comprises two palmitoyl residues in the N and in the C terminal regions of the peptide.

In some embodiments of the invention, a liposomal vaccine composition comprises:
a) a peptide antigen displayed on the surface of the liposome, which comprises, consists essentially of or consists of SEQ ID NO: 2, SEQ ID NO: 3, SEQ ID NO: 4 or SEQ ID NO: 12;
b) a peptide comprising a T-cell epitope; and
c) an adjuvant,
wherein the peptide antigen comprises one or two palmitoyl residues in the N terminal regions of the peptide.

In some embodiments of the invention, a liposomal vaccine composition comprises:
a) a peptide antigen displayed on the surface of the liposome, which comprises, consists essentially of or consists of SEQ ID NO: 2,
b) a peptide comprising a T-cell epitope; and
c) an adjuvant,
wherein the peptide antigen comprises one or two palmitoyl residues in the N terminal regions of the peptide.

In some embodiments of the invention, a liposomal vaccine composition comprises:
a) a peptide antigen displayed on the surface of the liposome, which comprises, consists essentially of or consists of SEQ ID NO: 12,
b) a peptide comprising a T-cell epitope; and
c) an adjuvant,
wherein the peptide antigen comprises one or two palmitoyl residues in the N terminal regions of the peptide.

In some embodiments of the invention, the liposomal vaccine composition comprises:
a. a peptide antigen displayed on the surface of the liposome, which comprises, consists essentially of or consists of an amino acid sequence selected from the group consisting of SEQ ID NO: 1, SEQ ID NO: 38, and SEQ ID NO: 45,
b. a peptide comprising a T-cell epitope; and
c. an adjuvant,
wherein the adjuvant comprises at least one of a TLR4 ligand and/or a TLR9 ligand.

In some embodiments of the invention, the liposomal vaccine composition comprises:
a. a peptide antigen displayed on the surface of the liposome, which comprises, consists essentially of or consists of an amino acid sequence selected from the group consisting of SEQ ID NO: 1, SEQ ID NO: 38, and SEQ ID NO: 45,
b. a peptide comprising a T-cell epitope which comprises, consists essentially of or consists of an amino acid sequence selected from the group consisting of SEQ ID NO: 24, SEQ ID NO: 25, SEQ ID NO: 26, or SEQ ID NO: 27 or any combination thereof, and
c. an adjuvant,
wherein the adjuvant comprises at least one of a TLR4 ligand and/or a TLR9 ligand.

In some embodiments of the invention, the liposomal vaccine composition comprises:
a. a peptide antigen displayed on the surface of the liposome, which comprises, consists essentially of or consists of an amino acid sequence selected from the group consisting of SEQ ID NO: 1, SEQ ID NO: 38, and SEQ ID NO: 45,
b. a peptide comprising a T-cell epitope which comprises, consists essentially of or consists of an amino acid sequence SEQ ID NO: 24 and SEQ ID NO: 25 and SEQ ID NO: 26 and SEQ ID NO: 27.
c. an adjuvant,
wherein the adjuvant comprises at least one of a TLR4 ligand and/or a TLR9 ligand

In some embodiments of the invention, the liposomal vaccine composition comprises:
a. a peptide antigen displayed on the surface of the liposome, which comprises, consists essentially of or consists of an amino acid sequence selected from the group consisting of SEQ ID NO: 1, SEQ ID NO: 38, and SEQ ID NO: 45,
b. a peptide comprising a T-cell epitope which comprises, consists essentially of or consists of an amino acid sequence selected from the group consisting of SEQ ID NO: 46, SEQ ID NO: 25, SEQ ID NO: 26 or SEQ ID NO: 27.
c. an adjuvant.

In some embodiments of the invention, the liposomal vaccine composition comprises:
a. a peptide antigen displayed on the surface of the liposome, which comprises, consists essentially of or consists of an amino acid sequence selected from the group consisting of SEQ ID NO: 1, SEQ ID NO: 38, and SEQ ID NO: 45,
b. a peptide comprising a T-cell epitope which comprises, consists essentially of or consists of an amino acid sequence SEQ ID NO: 46 and SEQ ID NO: 25 and SEQ ID NO: 26 and SEQ ID NO: 27.
c. an adjuvant,
wherein the adjuvant comprises at least one of a TLR4 ligand and/or a TLR9 ligand.

In some embodiments of the invention, the liposomal vaccine composition comprises:
a. a peptide antigen displayed on the surface of the liposome, which comprises, consists essentially of or consists of an amino acid sequence selected from the group consisting of SEQ ID NO: 1, SEQ ID NO: 38, and SEQ ID NO: 45,
b. a peptide comprising a T-cell epitope which comprises, consists essentially of or consists of SEQ ID NO: 32 (SAT47) or a close sequence analogue of SEQ ID NO: 32; and
c. an adjuvant,
wherein the adjuvant comprises at least one of a TLR4 ligand and/or a TLR9 ligand.

In some embodiments of the invention, the liposomal vaccine composition comprises:
a. a peptide antigen displayed on the surface of the liposome, which comprises, consists essentially of or consists of an amino acid sequence selected from the group consisting of SEQ ID NO: 1, SEQ ID NO: 38, and SEQ ID NO: 45,
b. a peptide comprising a T-cell epitope which comprises, consists essentially of or consists of SEQ ID NO: 33 (SAT58); and
c. an adjuvant,
wherein the adjuvant comprises at least one of a TLR4 ligand and/or a TLR9 ligand.

In some embodiment of the invention, the liposomal vaccine composition comprises:
a. a peptide antigen displayed on the surface of the liposome, which comprises, consists essentially of or consists of an amino acid sequence selected from SEQ ID NO: 1, SEQ ID NO: 38, and SEQ ID NO: 45,
b. a peptide comprising a T-cell epitope which comprises, consists essentially of or consists of the amino acid sequence of SEQ ID NO: 33 (SAT58); and
c. an adjuvant; wherein the adjuvant comprises a toll-like receptor 4 ligand and/or a toll-like receptor 9 ligand. Optionally, the toll-like receptor 4 ligand comprises monophosphoryl lipid A (MPLA) and the toll-like receptor 9 ligand comprises CpG.

In some embodiments of the invention, a liposomal vaccine composition comprises:
a) A peptide antigen displayed on the surface of the liposome, which comprises, consists essentially of or consists of SEQ ID NO: 1, SEQ ID NO: 38, and SEQ ID NO: 45;
b) A peptide comprising a T-cell epitope, which comprises, consists essentially of or consists of SEQ ID NO: 32 (SAT47) or a close sequence analogue of SEQ ID NO: 32;
c) A TLR4 ligand comprising monophosphoryl lipid A (MPLA); and
d) A TLR9 ligand comprising CpG.

In some embodiments of the invention, a liposomal vaccine composition comprises:
a) A peptide antigen displayed on the surface of the liposome, which comprises, consists essentially of or consists of SEQ ID NO: 1, SEQ ID NO: 38, and SEQ ID NO: 45;
b) A peptide comprising a T-cell epitope; and
c) An adjuvant,
wherein the peptide antigen comprises one or two palmitoyl residues in the N and/or in the C terminal regions of the peptide.

The liposomal vaccine compositions of the invention can be administered a single time to the subject to generate a protective immune response. However, in some embodiments, the vaccine compositions of the invention are administered multiple times to the same subject. Thus, so-called prime-boost regimens may be employed according to the invention. Administration of the vaccine is typically separated by an intervening period of at least 1 week and often around 1-12 months.

The timing of administration (e.g. by injection) can vary significantly from once a day, to once a year, to once a decade. A typical regimen consists of an immunization followed by booster administration (e.g. by injection) at regular time intervals, such as 4- to 6-week intervals. However, less regular booster administration, such as annual boosting may be preferred for reasons of convenience and compliance.

One or more immunizations can be administered. A typical regimen consists of an immunization followed by booster injections at time intervals, such as 4-6 weeks intervals. Another regimen may consist of an immunization followed by booster injections 1, 2, 6, 9 and/or 12 months later.

Alternatively, booster injections can be on an irregular basis as indicated by monitoring of immune response (e.g. when the level of the antibodies is below a threshold determined by a doctor or a person skilled in the art).

The vaccine compositions of the invention represent a powerful new therapeutic option for prevention and treatment of a disease, disorder or abnormality associated with alpha-synuclein aggregates including, but not limited to, Lewy bodies and/or Lewy neurites and/or glial cytoplasmic inclusions, such as Parkinson's disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease. In some embodiments, the same vaccine composition is administered each time—a homologous vaccination regimen. Homologous vaccination refers to an immunization regimen using the same vaccine for both the prime (first immunization) and boost (second or any further immunization).

The vaccine compositions of the invention may be administered to the subject by any appropriate route of administration. As the skilled person would be aware, vaccine compositions may be administered by topical, oral, rectal, nasal or parenteral (such as intravenous, intradermal, subcutaneous, or intramuscular) routes. In addition, vaccine compositions may be incorporated into sustained release matrices such as biodegradable polymers, the polymers being implanted in the vicinity of, or in close proximity to, where delivery is desired. In preferred embodiments, the vaccine composition is administered intramuscularly or subcutaneously.

The vaccine compositions of the invention are administered to subjects in order to treat, prevent, induce a protective immune response against or alleviate the symptoms associated with a disease, disorder or abnormality associated with alpha-synuclein aggregates.

The vaccine compositions can thus have both prophylactic and therapeutic applications. The subject is a mammal and typically a human.

In an embodiment of the invention the disease, disorder or abnormality associated with alpha-synuclein aggregates is a synucleinopathy.

In other embodiments of the invention, the disease, disorder or abnormality associated with alpha-synuclein aggregates, or the synucleinopathy, may be selected from the group consisting of: the Parkinson's disease (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), diffuse Lewy body disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, and Down syndrome, multiple system atrophy (Shy-Drager syndrome, striatonigral degeneration and olivopontocerebellar atrophy), traumatic brain injury, chronic traumatic encephalopathy, dementia puglistica, tauopathies (Pick's disease, frontotemporal dementia, progressive supranuclear palsy, corticobasal degeneration and Niemann-Pick type C1 disease, frontotemporal dementia with Parkinsonism linked to chromosome 17), motor neuron disease, Huntington's disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Creutzfeldt-Jakob disease, ataxia telangiectatica, Meige's syndrome, subacute sclerosing panencephalitis, Gerstmann-Straussler-Scheinker disease, inclusion-body myositis, Gaucher disease, Krabbe disease as well as other lysosomal storage disorders (including Kufor-Rakeb syndrome and Sanfilippo syndrome) and rapid eye movement (REM) sleep behavior disorder.

In a preferred embodiment the disease, disorder or abnormality associated with alpha-synuclein aggregates, or the synucleinopathy, is selected from the group consisting of Lewy bodies and/or Lewy neurites and/or glial cytoplasmic inclusions, such as Parkinson's disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease. In an embodiment, the disease, disorder or abnormality associated with alpha-synuclein aggregates, or the synucleinopathy, is Parkinson's disease or Multiple System Atrophy, more preferably Multiple System Atrophy.

Accordingly, the invention provides a method of treating, preventing, inducing a protective immune response against or alleviating the symptoms associated with alpha-synuclein aggregates in a subject, the method comprising administering a vaccine composition of the invention to the subject.

Such methods may also be expressed in the form of a medical use of the vaccine compositions of the invention. Accordingly, the invention also provides a vaccine composition of the invention for use in treating, preventing, inducing a protective immune response against or alleviating the symptoms associated with a disease, disorder or abnormality associated with alpha-synuclein aggregates in a subject.

Thus, the invention further includes administering to the subject the liposomal vaccine composition or the pharmaceutical composition according to the invention for inducing an immune response against alpha-synuclein aggregates. In some embodiments the invention further includes administering to the subject the liposomal vaccine composition or the pharmaceutical composition according to the invention for inducing an immune response against alpha-synuclein protein. In some embodiments, administering to the subject the liposomal vaccine composition or the pharmaceutical composition according to the invention induces a protective immune response against alpha-synuclein aggregates. In some embodiments, administering to the subject the liposomal vaccine composition or the pharmaceutical composition according to the invention induces an immune response against alpha-synuclein protein, preferably human alpha-synuclein protein.

Similarly, the invention provides for the use of the vaccine compositions of the invention in the manufacture of a medicament for use in treating, preventing, inducing a protective immune response against or alleviating the symptoms associated with a disease, disorder or abnormality associated with alpha-synuclein aggregates in a subject.

It is an object of the present invention to provide a medicament for use in preventing and treating synucleinopathies in the form of a vaccine. It is a further object to provide vaccination peptides suitable for human use.

It is an object of the present invention to provide a liposomal composition for use in preventing and treating synucleinopathies in the form of a vaccine. It is a further object to provide antigenic peptides suitable for human use.

All embodiments herein apply to such methods or medical uses, however expressed. Administration of a vaccine composition of the invention to the subject results in the production of, typically polyclonal, IgG antibodies that bind to pathological and/or aggregated forms of alpha-synuclein. As already explained, those pathological and/or aggregated forms of alpha-syunclein comprise multimers. The antibodies produced may therefore be termed “alpha-synuclein-multimer specific” antibodies.

The present invention provides Liposomal vaccine compositions comprising the antigenic peptide in a therapeutically effective amount. The term “therapeutically effective amount” refers to the amount of antigenic/immunogenic composition which, when administered to a human or animal, elicits an immune response. The effective amount is readily determined by one of skill in the art following routine procedures.

In absolute amounts, it is preferred to use an amount of the antigenic peptide in the dose of at least 10 μg, for example at least 50 μg. In this connection it is important to note that the “μg peptide” referred to in the present invention refers to the amount of antigenic peptide in the dose and does not include other components of the liposomal vaccine composition.

The invention further provides kits containing vaccine compositions according to the invention. Accordingly, there is provided a kit for treating, preventing, inducing a protective immune response against or alleviating the symptoms associated with a disease, disorder or abnormality associated with alpha-synuclein aggregates in a subject comprising a liposomal vaccine composition of the invention as described herein. Such kits may be provided with suitable instructions for use. The instructions for use may explain the administration schedule for the compositions. The kits may therefore comprise multiple (separate) doses of the vaccine compositions of the invention. The instructions for use may further explain the storage conditions for the compositions, particularly during the time period between administration of the doses of the vaccine compositions. These kits may be applied to all relevant methods of the invention as disclosed herein.

Methods for producing liposomal vaccine compositions of the invention may rely upon crossflow injection, as exemplified herein. Such methods may comprise the following steps:
a) Dissolving the lipids (and adjuvant, if lipid based) that form the liposome in solution
b) Dissolving the peptide comprising a (universal) T-cell epitope in solution
c) Mixing the solutions from steps a. and b. using a crossflow injection module to form intermediate liposomes which encapsulate the peptide comprising a universal T-cell epitope
d) Extruding the intermediate liposomes through a membrane to reduce their size and polydispersity
e) Mixing a solution comprising a peptide antigen as described herein with the solution from step d using a crossflow injection module, resulting in insertion of the peptide antigen into the lipid bilayer of the liposomes.

Such methods are exemplified herein, which details may be applied to these aspects of the invention. In general terms, the methods use crossflow injection to encapsulate the peptide comprising a universal T-cell epitope and to insert the alpha-synuclein-derived peptide antigen into the lipid bilayer of the liposomes.

### Definitions

By “T-cell epitope” is meant an epitope that is specific to T-cells that are present in the majority of the human population. They commonly originate from antigens to which humans are normally exposed during their lifetime. Examples include antigens incorporated in routinely administered vaccines. Specific examples of T-cell epitopes include, but are not limited to, tetanus, influenza and diphtheria (including non-toxic mutants thereof such as CRM197), Keyhole limpet hemocyanin (KLH), Epstein Barr virus (EBV) and PaDre (pan HLA DR-binding Epitope). The ability of a T-cell epitope to activate T cells is the result of at least two complementary properties: i) affinity of binding to the HLA groove, meaning the strength of the binding, as well as ii) its capacity to bind different HLA haplotypes in a promiscuous manner, meaning the ability to cover very diverse human populations, with regards to the differences in the expression of HLA molecules. The T-cell epitopes may bind to a majority of MHC class II alleles present in the human population. The T-cell epitopes included in the vaccine compositions of the invention may thus be capable of stimulating a CD4 T-cell response. The T-cell epitopes included in the vaccine compositions of the invention may thus be capable of stimulating a helper T-cell response that enhances alpha-synuclein related antibody production by B-cells. They may be referred to as “universal” T-cell epitopes herein, a term commonly used in the art.

T-cell epitope herein is therefore to be understood as an epitope capable of stimulating a helper T-cell or T-cell helper response.

A “close sequence analogue” of the invention may include one or more modifications, such as, but not limited to amino acid substitutions, post-translational modification, extensions (addition of additional amino acids), shortening the length (removal of some amino acids), of a herein described sequence. In a preferred embodiment, a close sequence analogue of the T-cell epitopes of the inventions include one to five amino acid modifications, preferably one, two or three modifications.

The term “toll-like receptor 4 agonist” or “TLR4” refers to any compound that acts as an agonist of TLR4. Any suitable TLR4 agonist known to, or discovered by, those skilled in the art can be used in the invention. Examples of TLR4 ligands useful for the invention include TLR4 agonists, including, but not limited to, monophosphoryl lipid A (MPLA).

The term “toll-like receptor 9 agonist” or “TLR9” refers to any compound that acts as an agonist of TLR9. Any suitable TLR9 agonist known to, or discovered by, those skilled in the art can be used in the invention. Examples of TLR9 ligands useful for the invention include TLR9 agonists including, but not limited to, CpG oligonucleotides.

Pharmaceutically acceptable carriers, diluents, adjuvants and excipients are well known in the pharmaceutical art and are described, for example, in Remington's Pharmaceutical Sciences, 15^{th} or 18^{th} Ed. (Alfonso R. Gennaro, ed.; Mack Publishing Company, Easton, PA, 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc, 1999); Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); Fiedler's “Lexikon der Hilfstoffe” 5^{th} Ed., Edition Cantor Verlag Aulendorf 2002; “The Handbook of Pharmaceutical Excipients”, 4^{th} Ed., American Pharmaceuticals Association, 2003; and Goodman and Gilman's: the Pharmacological Basis of Therapeutics (Louis S. Goodman and Lee E. Limbird, eds.; McGraw Hill, 1992), the disclosures of which are hereby incorporated by reference.

The carriers, diluents, adjuvants and pharmaceutical excipients can be selected with regard to the intended route of administration and standard pharmaceutical practice. These compounds must be acceptable in the sense of being not deleterious to the recipient thereof. See Remington's Pharmaceutical Sciences, 15^{th} or 18^{th} Ed. (Alfonso R. Gennaro, ed.; Mack Publishing Company, Easton, PA, 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc, 1999); Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); Fiedler's “Lexikon der Hilfstoffe” 5^{th} Ed., Edition Cantor Verlag Aulendorf 2002; “The Handbook of Pharmaceutical Excipients”, 4^{th} Ed., American Pharmaceuticals Association, 2003; and Goodman and Gilman's: the Pharmacological Basis of Therapeutics (Louis S. Goodman and Lee E. Limbird, eds.; McGraw Hill, 1992), the disclosures of which are hereby incorporated by reference.

The term “liposomal vaccine composition” may be used interchangeably with “liposomal immunogenic composition” herein. A “close sequence analogue” of the invention may include one or more modifications, such as, but not limited to amino acid substitutions, post-translational modification, extensions (addition of additional amino acids), shortening the length (removal of some amino acids), of a herein described sequence.

Where an amino acid is referred to herein, it is typically the natural amino acid. However, the reference to an amino acid also encompasses non-natural amino acids (e.g., beta-amino acids, gamma-amino acids, D-amino acids) and, in the context of an overall peptide, a combination of natural and non-natural amino acids.

The native sequence of human alpha-synuclein is: MDVFMKGLSKAKEGVVAAAEKTKQGVAEAAGKTKEGVLYVGSKTKEGVVHGVA TVAEKTKEQVTNVGGAVVTGVTAVAQKTVEGAGSIAAATGFVKKDQLGKNEEGAP QEGILEDMPVDPDNEAYEMPSEEGYQDYEPEA (SEQ ID NO: 28).

An “immune response” involves the production of anti-aSyn antibodies, preferably of antibodies that specifically bind to alpha-synuclein. The production of such antibodies can be tested by any suitable method, such as an immunoassay, and specifically by ELISA.

### BRIEF DESCRIPTION OF DRAWINGS

FIG. 1 is a graph showing IgG titers against SEQ ID NO:2 peptide in mice immunized with SEQ ID NO:2-liposomal or SEQ ID NO:2 conjugate vaccines. The Y axis shows anti-SEQ ID NO: 2 IgG titers (AU/mL) and the X axis shows time measured by days post immunization. The liposomal vaccine treated group (black circles) is compared to the conjugate vaccine treated group (black squares).

FIG. 2 is a graph showing IgG titers against a-syn protein in mice immunized with SEQ ID NO:2-liposomal or SEQ ID NO:2 conjugate vaccines. The Y axis shows anti-a-syn protein IgG titers (AU/mL) and the X axis shows time measured by days post immunization. The liposomal vaccine treated group (black circles) is compared to the conjugate vaccine treated group (black squares).

FIG. 3 is a graph showing IgG titers against a-syn aggregates in mice immunized with SEQ ID NO:2-liposomal or SEQ ID NO:2 conjugate vaccines. The Y axis shows anti-a-syn aggregates IgG titers (AU/mL) and the X axis shows time measured by days post immunization. The liposomal vaccine treated group (black circles) is compared to the conjugate vaccine treated group (black squares).

### EXAMPLES

The invention will be further understood with reference to the following non-limiting examples.

### Example 1. Vaccine Synthesis and Formulation

The vaccine is produced as follows in a three-step approach, i.e., preparation of intermediate liposome followed by integration of CpG-Chol to generate the fully adjuvanted liposomes and finally insertion of an antigenic peptide.

Intermediate liposomes: First, lipids (DMPG, DMPC, cholesterol and monophosphoryl Hexa-acyl Lipid A, 3-Deacyl, the first adjuvant) are dissolved in ethanol at 60° C. After complete dissolution, the lipid/ethanol solution is filtered through a 0.2 m pore size filter into an injection system preheated at 60° C. In a separate vessel, the peptide comprising a T-cell epitope (e.g. SAT47, or a close sequence analogue) is solubilized in 10 mM Histidine, 250 mM Sucrose. This solution is filtered through a 0.2 m pore size filter and heated to 40° C. The lipid/ethanol solution and the peptide comprising a T-cell epitope (e.g. SAT47 or a close sequence analogue) solution are then mixed using a crossflow injection module to form the intermediate liposomes, which are subsequently subjected to active cooling followed by size reduction using repeated extrusion cycles. Finally ultra-/diafiltration (UDF) is performed to remove ethanol. The intermediate liposomes are filtered through a 0.2 m pore size filter and stored at 4° C. until used.

CpG-Chol integration: The intermediate liposomes are diluted to a concentration of 1 mg/mL of lipid and warmed up to 60° C. The second adjuvant, CpG-Chol, is added to the liposomes dropwise. The liposomal dispersion is incubated at 60° C. for an additional 30 minutes whilst stirring. The fully adjuvanted liposomes are purified by UDF and then filtered through a 0.45 m followed by a 0.2 m pore size filter and stored at 4° C.

Alpha-synuclein (Asyn) derived antigenic peptide insertion: The fully adjuvanted liposomes are diluted to a desired concentration of lipid. Concomitantly, the palmitoylated antigenic peptide (such as a peptide having the amino acid sequence of SEQ ID NO: 2, SEQ ID NO: 3 or SEQ ID NO: 4) is dissolved to a final concentration between 0.1 and 10 mg/mL of peptide. The liposomes and the peptide solution are finally mixed using a crossflow module and the liposomal dispersion is incubated at temperature between 30° C. and 60° C. under stirring. A UDF step is performed to exchange the buffer to the formulation system. The product is concentrated down to its final volume, filtered through a 0.45 m pore size filter followed by a 0.2 m pore size filter. The bulk drug product is stored at 4° C.

### Example 2: Immunogenicity Studies in Mice

Immunogenicity studies of vaccines displaying different alpha synuclein derived antigenic peptides conjugated to CRM197 through a cysteine, including the native alpha synuclein sequence and derivatives thereof (VARIOTOPES), were performed in BALB/c mice. Mice received a total of three subcutaneous (s.c.) immunizations (10 μg net peptide per injection) at days 0, 14 and 28. For this purpose 200 μl of the individual vaccines were applied in the flank of mice using an insulin 20 syringe with a G30-gauge needle. Blood was taken two weeks after the last injection To measure alpha synuclein-specific IgG titres by ELISA. For this, 96-well plates (Nunc-Maxisorp) were coated with recombinant human alpha synuclein (1 g/ml) and titers were calculated as EC50-values with PRISM® 5.04 (GraphPad Inc, San Diego, CA) by non-linear regression analysis (four-parameter logistic fit function).

The immunogenicity of vaccines containing peptides with serine exchanges along the alpha synuclein 113-124 native sequence was evaluated in BALB/c mice and compared to the immunogenicity of the vaccine containing the native sequence for direct comparison (Table 2).

TABLE 2
Treatment groups and the peptide sequences
present in individual vaccines are shown.
Group one was immunized with the vaccine containing
the native sequence for direct comparison.

| Group | Sequence |
|---|---|
| 1 | LEDMPVDPDNEA |
| | (SEQ ID NO: 38) |
| 2 | LESMPVDPDNES |
| | (SEQ ID NO:4) |
| 3 | SESMPVDPDNEA |
| | (SEQ ID NO:9) |
| 4 | LESSPVDPDNEA |
| | (SEQ ID NO: 39) |
| 5 | SEDMPVDPDNES |
| | (SEQ ID NO: 40) |
| 6 | LEDSPVDPDNES |
| | (SEQ ID NO: 41) |
| 7 | SEDSPVDPDNEA |
| | (SEQ ID NO: 42) |

Vaccines containing peptides with either serine exchanges at position 1 and 3 (group 3) or at position 3 and 12 (group 2) induced 4 to 6 times higher alpha synuclein-specific IgG responses than the vaccine containing the native peptide sequence (group 1). Vaccines containing peptides with serine exchanges at position 4 (groups 4, 6 and 7) did not increase the immunogenicity as significantly relative to the vaccine containing the native sequence (Table 3). A vaccine containing peptides with serine exchanges at positions 1 and 12 (group 5) increased the immunogenicity less than two-fold relative to the vaccine containing the native sequence (Table 3).

TABLE 3
The titers to alpha synuclein were evaluated in single
mice and values represent medians relative to the
median obtained with the native sequence.

| Group | Titres relative to group 1 in % |
|---|---|
| 1 | 100 |
| 2 | 622 |
| 3 | 397 |
| 4 | 146 |
| 5 | 189 |
| 6 | 110 |
| 7 | 101 |

As vaccines containing peptides with amino acid exchanges to serine at position 1, 3, and 12 were shown to induce high alpha synuclein-specific IgG titers, further immunogenicity study was performed using vaccines containing peptides with either a serine or a lysine exchange at position 1, 3, and 12. Some of the peptides tested were extended at their N-terminus by adding two glycine residues (Table 4). Furthermore, these vaccines were compared to a vaccine containing the previously selected antigenic peptide sequence DQPVLPD (SEQ ID NO: 43) for direct comparison.

TABLE 4
Treatment groups and the peptide sequences
present in individual vaccines are shown.
Group one was immunized with the vaccine
containing the peptide sequence LESMPVDPDNES
(group 2 Table 2) for bridging this experiment
to the previous experiment.

| Group | Sequence |
|---|---|
| 1 | LESMPVDPDNES |
| | (SEQ ID NO: 4 |
| 2 | GG-KEDMPVDPDNEA |
| | (SEQ ID NO: 3) |
| 3 | LEKMPVDPDNES |
| | (SEQ ID NO: 15) |
| 4 | SEKMPVDPDNEA |
| | (SEQ ID NO: 14) |
| 5 | LEKMPVDPDNEK |
| | (SEQ ID NO: 17) |
| 6 | GG-KESMPVDPDNEA |
| | (SEQ ID NO: 2) |
| 7 | GG-KESMPVDPDNEK |
| | (SEQ ID NO: 19) |
| 8 | DQPVLPD |
| | SEQ ID NO: 43 |

All newly designed vaccines induced higher alpha synuclein-specific IgG titres (up to three-fold—group 6) compared to the already highly immunogenic vaccine containing the antigenic peptide LESMPVDPDNES. Furthermore, all vaccines of group 1 to 7 described in Table 4 elicited substantially higher alpha synuclein-specific titers than the vaccine containing the antigenic peptide DQPVLPD (SEQ ID NO: 43) (Table 5).

TABLE 5
The titers to alpha synuclein were evaluated in single mice and
values represent medians relative to the median obtained with
the vaccine containing the antigenic peptide LESMPVDPDNES.

| Group | Titres relative to group 1 in % |
|---|---|
| 1 | 100 |
| 2 | 160 |
| 3 | 142 |
| 4 | 143 |
| 5 | 258 |
| 6 | 295 |
| 7 | 205 |
| 8 | 37 |

Unless defined otherwise, all technical and scientific terms used herein have the same meanings as commonly understood by one of ordinary skill in the art to which this invention belongs. All publications and patents specifically mentioned herein are incorporated by reference in their entirety for all purposes in connection with the invention.

### Example 3: Immunogenicity Study of SEQ ID NO: 2 Liposomal Vaccine and SEQ ID NO: 2 Conjugate Vaccine in Mice

The immunogenicity of a liposomal vaccine containing SEQ ID NO: 2 as antigenic peptide, (referred to as “SEQ ID NO: 2 liposomal vaccine”, and a protein-conjugate vaccine containing SEQ ID NO:2 as antigenic peptide coupled to the protein carrier CRM197 (referred to as “SEQ ID NO: 2 conjugate vaccine”) was evaluated in C57BL/6J female mice.

### a) SEQ ID NO: 2 Liposomal Vaccine Synthesis and Formulation

The vaccine is produced as follows in a three-step approach, according to Example 1. Intermediate liposomes: first, lipids (DMPG, DMPC, cholesterol and monophosphoryl Hexa-acyl Lipid A, (3D-(6-acyl) PHAD™ (Avanti Polar Lipids, USA)), the first adjuvant) were dissolved in ethanol at 60° C. After complete dissolution, the lipid/ethanol solution is filtered through a 0.2 m pore size filter into an injection system preheated at 60° C. In a separate vessel, the peptide comprising T-cell epitope SAT58 was dispersed in ethanol at room temperature by the aid of sonication and solubilized by dilution with 10 mM Histidine, 250 mM Sucrose. The SAT58 solution was filtered through a 0.2 m pore size filter and heated up to 40° C. The lipid/ethanol solution and the SAT58 solution were then mixed using a crossflow injection module to form the intermediate liposomes, which were subsequently subjected to active cooling followed by size reduction using repeated extrusion cycles. Finally ultra-/diafiltration (UDF) was performed to remove ethanol. The intermediate SAT58 liposomes were filtered through a 0.2 m pore size filter and stored at 4° C. until used.

CpG-Chol integration: The intermediate liposomes were diluted to a concentration of 1 mg/mL of lipid in 20 mM Histidine, 145 mM NaCl and warmed up to 60° C. The second adjuvant, CpG-Cholesterol, was added to the liposomes dropwise. The liposomal dispersion was incubated at 60° C. for an additional 30 minutes whilst stirring. The fully adjuvanted liposomes were purified by UDF and then filtered through a 0.45 m followed by a 0.2 m pore size filter and stored at 4° C.

Alpha-synuclein (Asyn) derived antigenic peptide insertion: The fully adjuvanted liposomes were diluted to a concentration of 1 mg/mL of total lipid content in 20 mM Histidine, 145 mM NaCl. Concomitantly, the N-terminal di-palmitoylated SEQ ID NO:2 peptide was dissolved to a final concentration of 1 mg/mL in 20 mM Histidine, 145 mM NaCl at 60° C. and the solution was filtered through a 0.2 m pore size filter. The liposomes and the peptide solution were finally mixed using a crossflow module and the liposomal dispersion was incubated at temperature of 60° C. under stirring for 30 minutes. A UDF step was performed to exchange the buffer to the final formulation system i.e. with 10 mM Histidine, 250 mM Sucrose buffer. The product was concentrated down to its final volume, filtered through a 0.45 m pore size filter followed by a 0.2 m pore size filter. The bulk drug product was stored at 4° C.

### b) SEQ ID NO: 2 Conjugate Vaccine Synthesis and Formulation:

The SEQ ID NO: 2 conjugate vaccine is a conjugate of the antigenic peptide SEQ ID NO: 2 to the carrier protein CRM197. The antigenic peptide is modified so as that it contains a cysteine residue. The antigenic peptide is coupled to the carrier protein CRM197 through the cysteine residue. The conjugation is a directed procedure using the side chain amino groups of lysine residues in CRM197 and the free thiol group of the amino (N)-terminal cysteine in the peptide. For the activation of CRM197, the aqueous CRM197 solution is adjusted to 10 mM phosphate buffered saline (PBS) and is then gently shaken with the bifunctional linker 4-maleimidobutyric acid N-hydroxysuccinimide ester (GMBS). Subsequently, excess of unreacted GMBS is removed by either dialysis or ultrafiltration. The obtained activated CRM197 solution is subsequently incubated with antigenic peptide (SEQ ID NO: 2) dissolved in phosphate buffer. The free thiol group of the cysteine within the peptide reacts with the maleimido group forming the final antigenic peptide (SEQ ID NO: 2)-CRM197 product.

### c) Study Design

A total of 20 C57BL/6J female mice, approximately 10 weeks old at 1^{t} immunization, were allocated to two groups (10 mice in group 1 and 10 mice in group 2) as indicated in Table 6. The two groups were immunized three times by subcutaneous (s.c) injection into the subcutis of the dorsum on Days 1, 15 and 29 with either SEQ ID NO:2 conjugate vaccine (group 1) or SEQ ID NO: 2 liposomal vaccine (group 2).

TABLE 6
study design

| | | Target Peptide | | | |
|---|---|---|---|---|---|
| Group | | Dose Level | Dose volume | Route of | |
| number | Vaccine | (μg/injection) | (mL/animal) | administration | Animals/group |
| 1 | SEQ ID NO: 2 | 10 | 0.2 | s.c | 10 |
| | conjugate vaccine | | | | |
| 2 | SEQ ID NO: 2 | 80 | 0.2 | s.c | 10 |
| | liposomal vaccine | | | | |

s.c: subcutaneous

Dose selection for the SEQ ID NO: 2 liposomal vaccine and the SEQ ID NO: 2 conjugate vaccine for this study in C57BL/6J mice is based on peak antibody levels in mice.

### d) Immunogenicity Results in Plasma:

Immunogenicity against SEQ ID NO:2 peptide, a-syn full-length human protein and a-syn aggregates was assessed in plasma samples collected on day 1 (pre-dose before the first administration) and one week after each immunization (on Days 8, 22 and 36). The a-syn aggregates were prepared according to the protocol described by Kumar et al. 2020.

The anti-SEQ ID NO: 2, anti-a-syn protein or anti-a-syn aggregates IgG titers were analyzed at each timepoint by an enzyme-linked immune sorbent assay (ELISA). Briefly, BSA conjugated SEQ ID NO: 2 peptide or a-syn protein or a-syn aggregates were immobilized on 96-well micro titers plates overnight. After washing and blocking, plates were incubated with the plasma samples for two hours at 37° C., allowing the antibodies present in plasma to bind the peptide or the proteins. After incubation, the plates were washed to remove non-reactive plasma components. The antibody/antigen complex was detected via a secondary anti-mouse IgG antibody conjugated to alkaline phosphatase. pNPP (p-Nitrophenyl Phosphate) substrate was added to the wells and optical density was read at 405 nM in an ELISA plate reader. The anti-SEQ ID NO: 2 peptide, anti-a-syn protein or anti-a-syn aggregate IgG titers were back-calculated against a calibration curve in eight two-fold serial dilution, using an unweighted four-parameter logistic regression model using the Gen5 software (BioTek, Switzerland). Results are expressed as AU/mL.

Data are expressed as geometric mean 95% confident interval (CI) overtime, with n=10/group (n=9 for group 2 on Day 22 and 36). 2-Way ANOVA with an uncorrected Fisher's LSD multiple comparison test was used for statistical analysis. *=p<0.05, **=p<0.01, ***=p<0.001, ****=p<0.0001.

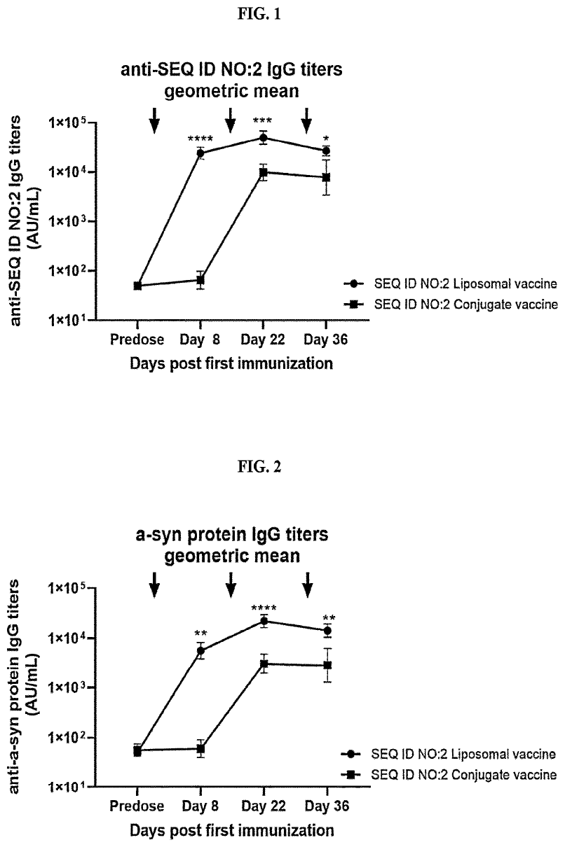

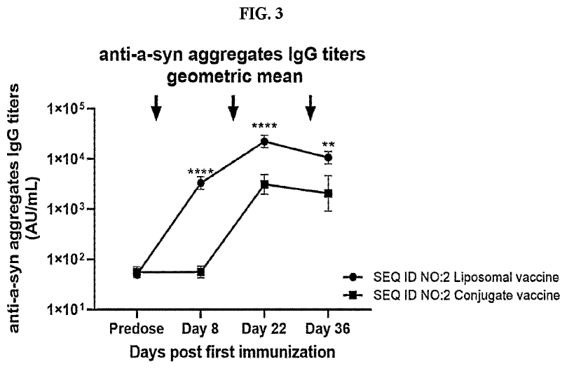

Results (FIGS. 1, 2, 3) showed that animals immunized with SEQ ID NO:2 liposomal vaccine developed a robust anti-SEQ ID NO:2 peptide, anti-a-syn protein and anti-a-syn aggregate response after one immunization, whereas two immunizations were required for the mice immunized with SEQ ID NO:2 conjugate vaccine. At all timepoints, SEQ ID NO:2 liposomal vaccine induced statistically significantly higher titers than SEQ ID NO:2 conjugated vaccine for all the readouts tested.

To conclude, the results confirm the potential of SEQ ID NO: 2 liposomal vaccine to induce a strong immune response in vivo against alpha-synuclein proteins and aggregated alpha-synuclein. Furthermore the results show the SEQ ID NO: 2 liposomal vaccine to provide a surprisingly increased immune response in vivo against anti-SEQ ID NO:2 peptide, alpha-synuclein proteins and aggregated alpha-synuclein compared to the SEQ ID NO:2 conjugate vaccine.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Moreover, all aspects and embodiments of the invention described herein are considered to be broadly applicable and combinable with any and all other consistent embodiments, including those taken from other aspects of the invention (including in isolation) as appropriate.

### REFERENCES

Alexander J, Sidney J, Southwood S, Ruppert J, Oseroff C, Maewal A, Snoke K, Serra H M, Kubo R T, Sette A, et al. Development of high potency universal D R-restricted helper epitopes by modification of high affinity D R-blocking peptides. Immunity
Andreas Puschmann, Roongroj Bhidayasiri, William J. Weiner, Synucleinopathies from bench to bedside, Parkinsonism & Related Disorders, Volume 18, Supplement 1, 2012, Pages S24-S27, ISSN 1353-8020
Askanas V, Engel W K, Alvarez R B, McFerrin J, Broccolini A. Novel immunolocalization of alpha-synuclein in human muscle of inclusion-body myositis, regenerating and necrotic muscle fibers, and at neuromuscular junctions. J Neuropathol Exp Neurol. 2000 July; 59(7):592-8.
Bassil F, Fernagut P O, Bezard E, Pruvost A, Leste-Lasserre T, Hoang Q Q, Ringe D, Petsko G A, Meissner W G. Reducing C-terminal truncation mitigates synucleinopathy and neurodegeneration in a transgenic model of multiple system atrophy. Proc Natl Acad Sci USA. 2016 Aug. 23; 113(34):9593-8
Braak H, Sastre M, Del Tredici K. Development of alpha-synuclein immunoreactive astrocytes in the forebrain parallels stages of intraneuronal pathology in sporadic Parkinson's disease. Acta Neuropathol. 2007 September; 114(3):231-41.
Bengoa-Vergniory N, Roberts R F, Wade-Martins R, Alegre-Abarrategui J. Alpha-synuclein oligomers: a new hope. Acta Neuropathol. 2017 December; 134(6):819-838.
Bernis M E, Babila J T, Breid S, Wusten K A, Wüllner U, Tamgüney G. Prion-like propagation of human brain-derived alpha-synuclein in transgenic mice expressing human wild-type alpha-synuclein. Acta Neuropathol Commun. 2015 Nov. 26; 3:75
Charles V., Mezey E., Reddy P. H., Dehejia A., Young T A., Polymeropoulos M. H., Brownstein M. J., Tagle D. A. (2000). Alpha-synuclein immunoreactivity of huntingtin polyglutamine aggregates in striatum and cortex of Huntington's disease patients and transgenic mouse models. Neurosci. Lett. 289, 29-32
Dickson D W, Crystal H, Mattiace L A, Kress Y, Schwagerl A, Ksiezak-Reding H, Davies P, Yen S H. Diffuse Lewy body disease: light and electron microscopic immunocytochemistry of senile plaques. Acta Neuropathol. 1989; 78(6):572-84.
Del Guercio M F, Jeff Alexander, Ralph T. Kubo, Thomas Arrhenius, Ajesh Maewal, Ettore Appella, Stephen L. Hoffman, Trevor Jones, Danila Valmori, Kazuyasu Sakaguchi, Howard M. Grey, Alessandro Sette, Potent immunogenic short linear peptide constructs composed of B cell epitopes and Pan D R T Helper Epitopes (PADRE) for antibody responses in vivo, Vaccine, Volume 15, Issue 4, 1997, Pages 441-448.
Dufty B M, Warner L R, Hou S T, Jiang S X, Gomez-Isla T, Leenhouts K M, Oxford J T, Feany M B, Masliah E, Rohn T T. Calpain-cleavage of alpha-synuclein: connecting proteolytic processing to disease-linked aggregation. Am J Pathol. 2007 May; 170(5):1725-38
FERMAN, T., BOEVE, B., SMITH, G., SILBER, M., LUCAS, J., GRAFF-RADFORD, N., IVNIK, R. (2002). Dementia with Lewy bodies may present as dementia and REM sleep behavior disorder without parkinsonism or hallucinations. Journal of the International Neuropsychological Society, 8(7), 907-914. doi:10.1017/S1355617702870047
Fleming S. M., Ashley Davis, Emily Simons, Targeting alpha-synuclein via the immune system in Parkinson's disease: Current vaccine therapies, Neuropharmacology, Volume 202, 2022, 108870, ISSN 0028-3908
Galvin J E, Lee V M, Trojanowski J Q. Synucleinopathies: Clinical and Pathological Implications. Arch Neurol. 2001; 58(2):186-190. doi:10.1001/archneur.58.2.186
Jellinger K A. Neuropathological spectrum of synucleinopathies. Mov Disord. 2003 September; 18 Suppl 6:S2-12. doi: 10.1002/mds.10557. PMID: 14502650.
Kosaka K. Diffuse Lewy body disease in Japan. J Neurol. 1990 June; 237(3):197-204.
Kovari E, Burkhardt K, Lobrinus J A, Bouras C. Lewy body dysphagia. Acta Neuropathol. 2007 September; 114(3):295-8. doi: 10.1007/s00401-007-0233-6. Epub 2007 Jun. 19. PMID: 17576582
Lashuel H A, Overk C R, Oueslati A, Masliah E. The many faces of α-synuclein: from structure and toxicity to therapeutic target. Nat Rev Neurosci. 2013 January; 14(1):38-48.
Lee H J, Suk J E, Bae E J, Lee J H, Paik S R, Lee S J. Assembly-dependent endocytosis and clearance of extracellular alpha-synuclein. Int J Biochem Cell Biol. 2008; 40(9):1835-49
Lippa C F, Schmidt M L, Lee V M, Trojanowski J Q. Antibodies to alpha-synuclein detect Lewy bodies in many Down's syndrome brains with Alzheimer's disease. Ann Neurol. 1999; 45:353-7
Mandler M, Valera E, Rockenstein E, Weninger H, Patrick C, Adame A, Santic R, Meindl S, Vigl B, Smrzka O, Schneeberger A, Mattner F, Masliah E. Next-generation active immunization approach for synucleinopathies: implications for Parkinson's disease clinical trials. Acta Neuropathol. 2014; 127(6):861-79.
Nimmo J T, Smith H, Wang C Y, Teeling J L, Nicoll J A R, Verma A, Dodart J C, Liu Z, Lin F, Carare R O. Immunisation with UB-312 in the Thy1SNCA mouse prevents motor performance deficits and oligomeric a-synuclein accumulation in the brain and gut. Acta Neuropathol. 2022 January; 143(1):55-73
McKee A C, Stern R A, Nowinski C J, Stein T D, Alvarez V E, Daneshvar D H, Lee H S, Wojtowicz S M, Hall G, Baugh C M, Riley D O, Kubilus C A, Cormier K A, Jacobs M A, Martin B R, Abraham C R, Ikezu T, Reichard R R, Wolozin B L, Budson A E, Goldstein L E, Kowall N W, Cantu R C. The spectrum of disease in chronic traumatic encephalopathy. Brain. 2013 January; 136(Pt 1):43-64. doi: 10.1093/brain/aws307. Epub 2012 Dec. 2. Erratum in: Brain. 2013 October; 136(Pt 10):e255. PMID: 23208308; PMCID: PMC3624697.
Panina-Bordignon P, Tan A, Termijtelen A, Demotz S, Corradin G, Lanzavecchia A. Universally immunogenic T cell epitopes: promiscuous binding to human MHC class II and promiscuous recognition by T cells. Eur J Immunol. 1989 December; 19(12):2237-42.
Pihlgren M., Silva A. B., Madani R., Giriens V., Waeckerle-Men Y., Fettelschoss A., Hickman D. T., L6pez-Deber M. P., Ndao D. M., Vukicevic M., Buccarello A L., Gafner V., Chuard N., Reis P., Piorkowska K., Pfeifer A., Kundig T. M., Muhs A., Johansen P., TLR4- and TRIF-dependent stimulation of B lymphocytes by peptide liposomes enables T eel/-independent isotype switch in mice. Blood. January 3; 121 (1):85-94 (2013).
Schmitz, M., Villar-Pique, A., Llorens, F. et al. Cerebrospinal Fluid Total and Phosphorylated α-Synuclein in Patients with Creutzfeldt-Jakob Disease and Synucleinopathy. Mol Neurobiol 56, 3476-3483 (2019). https://doi.org/10.1007/s12035-018-1313-4
Smith B R, Santos M B, Marshall M S, Cantuti-Castelvetri L, Lopez-Rosas A, Li G, van Breemen R, Claycomb K I, Gallea J I, Celej M S, Crocker S J, Givogri M I, Bongarzone E R. Neuronal inclusions of alpha-synuclein contribute to the pathogenesis of Krabbe disease. J Pathol. 2014; 232:509-521. doi: 10.1002/path.4328.
Spillantini M G, Schmidt M L, Lee V M, Trojanowski J Q, Jakes R, Goedert M. Alpha-synuclein in Lewy bodies. Nature. 1997 Aug. 28; 388(6645):839-40.
Usenovic M, Tresse E, Mazzulli J R, Taylor J P, Krainc D. Deficiency of ATP13A2 leads to lysosomal dysfunction, a-synuclein accumulation, and neurotoxicity. J Neurosci. 2012 Mar. 21; 32(12):4240-6. doi: 10.1523/JNEUROSCI.5575-11.2012. PMID: 22442086; PMCID: PMC3462811.
Volc D, Poewe W, Kutzelnigg A, LGhrs P, Thun-Hohenstein C, Schneeberger A, Galabova G, Majbour N, Vaikath N, El-Agnaf O, Winter D, Mihailovska E, Mairhofer A, Schwenke C, Staffler G, Medori R. Safety and immunogenicity of the α-synuclein active immunotherapeutic PDOlA in patients with Parkinson's disease: a randomised, single-blinded, phase 1 trial. Lancet Neurol. 2020 July; 19(7):591-600.
Wilhelmsen K C, Fornman M S, Rosen H J, Alving L I, Goldman J, Feiger J, et al. 17q-linked frontotemporal dementia-amyotrophic lateral sclerosis without tau mutations with tau and alpha-synuclein inclusions. Arch Neurol. 2004; 61(3):398-406
Winder-Rhodes S E, Garcia-Reitböck P, Ban M, et al. Genetic and pathological links between Parkinson's disease and the lysosomal disorder Sanfilippo syndrome. Mov Disord 2012; 27(2):312-315
Yamaguchi et al., J Neuropathol Exp Neurol. 2004, 80th annual meeting, vol. 63
Yuko Saito, M D, PHD, Kinuko Suzuki, M D, Christine M. Hulette, M D, Shigeo Murayama, M D, PHD, Aberrant Phosphorylation of α-Synuclein in Human Niemann-Pick Type C1 Disease, Journal of Neuropathology & Experimental Neurology, Volume 63, Issue 4, April 2004.
American Pharmaceuticals Association, 2003; and Goodman and Gilman's: the Pharmacological Basis of Therapeutics (Louis S. Goodman and Lee E. Limbird, eds.; McGraw Hill, 1992)
Fiedler's “Lexikon der Hilfstoffe” 5th Ed., Edition Cantor Verlag Aulendorf 2002; “The Handbook of Pharmaceutical Excipients”, 4th Ed
Handbook of Pharmaceutical Excipients, 3rd Ed. (Arthur H. Kibbe, ed.; Amer. Pharmaceutical Assoc, 1999)
Pharmaceutical Codex: Principles and Practice of Pharmaceutics 12th Ed. (Walter Lund ed.; Pharmaceutical Press, London, 1994)
Remington's Pharmaceutical Sciences, 15th or 18th Ed. (Alfonso R. Gennaro, ed.; Mack Publishing Company, Easton, PA, 1990)
Remington: the Science and Practice of Pharmacy 19th Ed. (Lippincott, Williams & Wilkins, 1995)

1. A liposomal vaccine composition comprising:
a. A peptide antigen displayed on the surface of the liposome;
b. A peptide comprising a T-cell epitope; and
c. An adjuvant;
wherein the peptide antigen comprises, consists essentially of or consists of the structure:
X₁-X₂-X₃-E-X₄-X₅-P-V-D-P-D-N-E-X₆,
wherein:
E is glutamic acid, P is proline; V is Valine, D is aspartic acid, N is asparagine;
X₁ is present or not and, if present, is G, wherein G is glycine;
X₂, is present or not and, if present, is G, wherein G is defined as above;
X₃ is L, K, or S, wherein L is leucine, K is lysine, and S is serine;
X₄ is D, K or S, wherein D, K and S are as defined above;
X₅ is M, wherein M is methionine or methionine sulfoxide;
X₆ is A, K or S, wherein A is alanine and K, and S are as defined above;
with the proviso that X₃-E-X₄-X₅-P-V-D-P-D-N-E-X₆ is not L-E-D-M-P-V-D-P-D-N-E-A,
and which comprises between 1 and 5 amino acid differences compared with the amino acid sequence G-I-L-E-D-M-P-V-D-P-D-N-E-A,
and wherein the peptide antigen does not comprise the dipeptide Y-E immediately following X₆, wherein Y is tyrosine and E is as defined above.

2. The liposomal composition of claim 1 wherein X₁ and X₂ are present.

3. The liposomal composition of claim 1 wherein X₁ and X₂ are absent.

4. The liposomal composition of any one of claims 1 to 3, wherein X₅ is methionine sulfoxide.

5. The liposomal composition of any one of claims 1 to 4, wherein X₆ is A or S.

6. The liposomal composition of any one of claims 1 to 5, wherein X₃ is L or K.

7. The liposomal composition of any one of claims 1 to 6, wherein X₄ is D or S.

8. The liposomal composition of claim 1 wherein the peptide antigen is selected from the group consisting of GGKESMPVDPDNEA (SEQ ID NO: 2), GGKEDMPVDPDNEA (SEQ ID NO: 3), LESMPVDPDNES (SEQ ID NO: 4), LEKMPVDPDNEA (SEQ ID NO: 5), GGSESMPVDPDNEA (SEQ ID NO: 6), GGSESMPVDPDNES (SEQ ID NO: 7), KEDMPVDPDNEA (SEQ ID NO: 8), SESMPVDPDNEA (SEQ ID NO: 9), LESMPVDPDNEA (SEQ ID NO: 10), GGKESMPVDPDNES (SEQ ID NO: 11), KESMPVDPDNEA (SEQ ID NO: 12), KESMPVDPDNES (SEQ ID NO: 13), SEKMPVDPDNEA (SEQ ID NO: 14), LEKMPVDPDNES (SEQ ID NO: 15), GGSESMPVDPDNEK (SEQ ID NO: 16), LEKMPVDPDNEK (SEQ ID NO: 17), GGKEKMPVDPDNEA (SEQ ID NO: 18), GGKESMPVDPDNEK (SEQ ID NO: 19) and GGKEKMPVDPDNEK (SEQ ID NO: 20).

9. The liposomal vaccine composition of claim 1, wherein the peptide antigen is selected from the group consisting of GGKESMPVDPDNEA (SEQ ID NO: 2), GGKEDMPVDPDNEA (SEQ ID NO: 3), LESMPVDPDNES (SEQ ID NO: 4) and KESMPVDPDNEA (SEQ ID NO: 12).

10. The liposomal vaccine composition of claim 1, wherein the peptide antigen consists of GGKESMPVDPDNEA (SEQ ID NO: 2).

11. The liposomal vaccine composition of claim 1, wherein the peptide antigen consists of KESMPVDPDNEA (SEQ ID NO: 12).

12. The liposomal vaccine composition of any one of the preceding claims, wherein the peptide antigen further comprises at least one chemical modification.

13. The liposomal vaccine composition of any one of the preceding claims, wherein the peptide comprising a T-cell epitope comprises at least one amino acid sequence selected from the group consisting of SEQ ID NO: 35 (PaDre), SEQ ID NO: 36 (P2), SEQ ID NO: 37 (P30), SEQ ID NO: 21 (SAT13), SEQ ID NO: 22 (SAT15), SEQ ID NO: 23 (SAT17), SEQ ID NO: 24, SEQ ID NO: 25, SEQ ID NO: 26, SEQ ID NO: 27 and/or any combination thereof.

14. The liposomal vaccine composition of any one of the preceding claims, wherein the peptide comprising a T-cell epitope comprises, consists essentially of or consists of an amino acid sequence selected from the group consisting of SEQ ID NO: 29 (SAT42), SEQ ID NO: 30 (SAT43), SEQ ID NO: 31 (SAT44), SEQ ID NO: 32 (SAT47), SEQ ID NO: 35 (PaDre), SEQ ID NO: 36 (P2) and SEQ ID NO: 37 (P30), or a close sequence analogue thereof.

15. The liposomal vaccine composition of any one of the preceding claims, wherein the peptide comprising a T-cell epitope comprises, consists essentially of or consists of the amino acid sequence of SEQ ID NO: 32 (SAT47) or a close sequence analogue thereof.

16. The liposomal vaccine composition of any of the preceding claims, wherein the peptide comprising a T-cell epitope comprises, consists essentially of, or consists of the amino acid sequence SEQ ID NO: 33 (SAT58).

17. The liposomal vaccine composition of any one of the preceding claims, wherein the adjuvant comprises a toll-like receptor 4 ligand and/or a toll-like receptor 9 ligand.

18. The liposomal vaccine composition of claim 17, wherein the toll-like receptor 4 ligand comprises monophosphoryl lipid A (MPLA) and/or wherein the toll-like receptor 9 ligand comprises CpG.

19. The liposomal vaccine composition of any one of the preceding claims, wherein the adjuvant comprises monophosphoryl lipid A (MPLA).

20. The liposomal vaccine composition of any one of the preceding claims, wherein the adjuvant further comprises CpG.

21. The liposomal vaccine composition of any one of the preceding claims, wherein the peptide comprising a T-cell epitope is encapsulated in the liposome.

22. A liposomal vaccine composition comprising:
d. a peptide antigen displayed on the surface of the liposome, which comprises, consists essentially of or consists of an amino acid sequence selected from SEQ ID NO: 2, SEQ ID NO 3, SEQ ID NO 4, SEQ ID NO 5 or SEQ ID NO:12,
e. a peptide comprising a T-cell epitope which comprises, consists essentially of, or consists of the amino acid sequence of SEQ ID NO: 24, and/or SEQ ID NO: 25, and/or SEQ ID NO: 27 and
f. an adjuvant; wherein the adjuvant comprises a toll-like receptor 4 ligand, preferably monophosphoryl lipid A (MPLA), and/or a toll-like receptor 9 ligand, preferably CpG.

23. A liposomal vaccine composition comprising:
g. a peptide antigen displayed on the surface of the liposome, which comprises, consists essentially of or consists of an amino acid sequence selected from SEQ ID NO: 2, SEQ ID NO 3, SEQ ID NO 4, SEQ ID NO 5 or SEQ ID NO:12,
h. a peptide comprising a T-cell epitope which comprises, consists essentially of, or consists of the amino acid sequence of SEQ ID NO: 46, and/or SEQ ID NO: 25, and/or SEQ ID NO: 27 and
i. an adjuvant; wherein the adjuvant comprises a toll-like receptor 4 ligand, preferably monophosphoryl lipid A (MPLA), and/or a toll-like receptor 9 ligand, preferably CpG.

24. The liposomal vaccine according to claim 22 or 23, wherein the peptide antigen comprises, consists essentially of, or consist of amino acid sequence SEQ ID NO: 2.

25. The liposomal vaccine according to claim 22 or 23, wherein the peptide antigen comprises, consists essentially of, or consist of amino acid sequence SEQ ID NO: 12

26. A liposomal vaccine composition comprising:
a. a peptide antigen displayed on the surface of the liposome, which comprises, consists essentially of or consists of an amino acid sequence selected from SEQ ID NO: 2, SEQ ID NO: 3, SEQ ID No: 4, SEQ ID NO: 5 or SEQ ID NO: 12; and
b. a peptide comprising a T-cell epitope which comprises, consists essentially of or consists of the amino acid sequence of SEQ ID NO: 32 (SAT47) or a close sequence analogue thereof; and
c. an adjuvant; wherein the adjuvant comprises a toll-like receptor 4 ligand; and wherein the toll-like receptor 4 ligand comprises monophosphoryl lipid A (MPLA).

27. A liposomal vaccine composition comprising:
a. a peptide antigen displayed on the surface of the liposome, which comprises, consists essentially of or consists of an amino acid sequence selected from SEQ ID NO: 2, SEQ ID NO: 3, SEQ ID No: 4, SEQ ID NO: 5 or SEQ ID NO: 12; and
b. a peptide comprising a T-cell epitope which comprises, consists essentially of or consists of the amino acid sequence of SEQ ID NO: 33 (SAT58); and
c. an adjuvant; wherein the adjuvant comprises a toll-like receptor 4 ligand; and wherein the toll-like receptor 4 ligand comprises monophosphoryl lipid A (MPLA).

28. The liposomal vaccine composition of claim 26 or 27, wherein the peptide antigen comprises, consists essentially of or consists of an amino acid sequence selected from SEQ ID NO: 2, SEQ ID NO: 3 SEQ ID NO: 4 or SEQ ID NO: 12.

29. The liposomal vaccine composition of claim 26 or 27, wherein the peptide antigen comprises, consists essentially of or consists of an amino acid sequence SEQ ID NO: 2.

30. The liposomal vaccine composition of claim 26 or 27, wherein the peptide antigen comprises, consists essentially of or consists of an amino acid sequence SEQ ID NO: 12.

31. A liposomal vaccine composition comprising:
a. a peptide antigen displayed on the surface of the liposome, which comprises, consists essentially of or consists of an amino acid sequence SEQ ID NO: 2,
b. a peptide comprising a T-cell epitope which comprises, consists essentially of or consists of the amino acid sequence of SEQ ID NO: 32 (SAT47) or a close sequence analogue thereof; and
c. an adjuvant; wherein the adjuvant comprises a toll-like receptor 4 ligand and/or a toll-like receptor 9 ligand; and wherein the toll-like receptor 4 ligand comprises monophosphoryl lipid A (MPLA), wherein the toll-like receptor 9 ligand comprises CpG.

32. A liposomal vaccine composition comprising:
a. a peptide antigen displayed on the surface of the liposome, which comprises, consists essentially of or consists of an amino acid sequence SEQ ID NO: 2,
b. a peptide comprising a T-cell epitope which comprises, consists essentially of or consists of the amino acid sequence of SEQ ID NO: 33 (SAT58) and
c. an adjuvant; wherein the adjuvant comprises a toll-like receptor 4 ligand and/or a toll-like receptor 9 ligand; and wherein the toll-like receptor 4 ligand comprises monophosphoryl lipid A (MPLA), wherein the toll-like receptor 9 ligand comprises CpG.

33. A pharmaceutical composition comprising the liposomal vaccine composition of any one of the claims 1 to 32, and a pharmaceutically acceptable carrier, diluent and/or excipient.

34. A kit comprising a liposomal vaccine composition as defined in any one of claims 1 to 32 or the pharmaceutical composition of claim 33 and a container.

35. The liposomal vaccine composition as defined in any one of claims 1 to 32, or pharmaceutical composition as defined in claim 33, or kit as defined in claim 34 for use in the treatment or prevention of diseases, disorders or abnormalities associated with alpha-synuclein aggregates.

36. The liposomal vaccine composition, pharmaceutical composition or kit for use of claim 35, wherein the disease, disorder or abnormality associated with alpha-synuclein aggregates is a synucleinopathy.

37. The liposomal vaccine composition, pharmaceutical composition or kit for use of claim 35 or 36, wherein the disease, disorder or abnormality associated with alpha-synuclein aggregates or the synucleinopathy is selected from the group consisting of Parkinson's disease (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), diffuse Lewy body disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, and Down syndrome, multiple system atrophy (Shy-Drager syndrome, striatonigral degeneration and olivopontocerebellar atrophy), traumatic brain injury, chronic traumatic encephalopathy, dementia puglistica, tauopathies (Pick's disease, frontotemporal dementia, progressive supranuclear palsy, corticobasal degeneration and Niemann-Pick type C1 disease, frontotemporal dementia with Parkinsonism linked to chromosome 17), motor neuron disease, Huntington's disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Creutzfeldt-Jakob disease, ataxia telangiectatica, Meige's syndrome, subacute sclerosing panencephalitis, Gerstmann-Straussler-Scheinker disease, inclusion-body myositis, Gaucher disease, Krabbe disease as well as other lysosomal storage disorders (including Kufor-Rakeb syndrome and Sanfilippo syndrome) and rapid eye movement (REM) sleep behavior disorder.

38. The liposomal vaccine composition, pharmaceutical composition or kit for use according to any one of claims 35 to 37, wherein the disease, disorder or abnormality associated with alpha-synuclein aggregates or the synucleinopathy is selected from the group consisting Lewy bodies and/or Lewy neurites and/or glial cytoplasmic inclusions, such as Parkinson's disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease.

39. The liposomal vaccine composition, pharmaceutical composition or kit for use according to any one of claims 35 to 37, wherein the disease, disorder or abnormality associated with alpha-synuclein aggregates or the synucleinopathy is Multiple System Atrophy.

40. The liposomal vaccine composition, pharmaceutical composition or kit for use according to claims 35 to 37, wherein the disease, disorder or abnormality associated with alpha-synuclein aggregates or the synucleinopathy is Parkinson's disease.

41. A method for prophylaxis, treatment and alleviation of diseases associated with the disease, disorder or abnormality associated with alpha-synuclein aggregates; wherein the method comprises administering to the subject the liposomal vaccine composition of any one of claims 1 to 32 or the pharmaceutical composition of claim 33.

42. The method of claim 41, wherein the disease, disorder or abnormality associated with alpha-synuclein aggregates is a synucleinopathy.

43. The method of any claim 41 or 42, wherein administering to the subject the liposomal vaccine composition of any one of claims 1 to 32 or the pharmaceutical composition of claim 33 induces an immune response against alpha-synuclein aggregates.

44. The method of any claim 41 or 42, wherein administering to the subject the liposomal vaccine composition of any one of claims 1 to 32 or the pharmaceutical composition of claim 33 induces a protective immune response against alpha-synuclein aggregates.

45. The method of any claim 41 or 42, wherein the administering to the subject the liposomal vaccine composition of any one of claims 1 to 32 or the pharmaceutical composition of claim 33 induces an immune response against alpha-synuclein protein, preferably human alpha-synuclein protein.
